(19) United States
(12) Patent Application Publication
(10) Pub. No.: US 2021/0292315 A1
(43) Pub. Date: Sep. 23, 2021

(54) SELENIUM-CONTAINING ISOXAZOLAMINE COMPOUND, PREPARATION METHOD THEREFOR, AND USE THEREOF

(71) Applicant: SHANGHAI XINGYE PHARMACEUTICAL TECHNOLOGY CO., LTD, Shanghai (CN)

(72) Inventors: Weibin Song, Shanghai (CN); Shuaishuai Ni, Shanghai (CN); Yinan Zhang, Shanghai (CN); Yanhui Liu, Shanghai (CN)

(21) Appl. No.: 17/261,955

(22) PCT Filed: Jul. 22, 2019
(86) PCT No.: PCT/CN2019/097093

§ 371 Date: Jan. 21, 2021

(30) Foreign Application Priority Data
Jul. 22, 2018 (CN) 201810808159.4

(51) Int. Cl. C07D 421/04; C07D 499/80; C07D 293/10; A61P 37/06

(52) U.S. Cl. CPC C07D 421/04; A61P 37/06; C07D 293/10; C07D 499/80

(57) ABSTRACT

The present invention disclosed a series of novel selenium-containing isoxazolamine derivatives as shown in formula I, which could regulate the generation and/or activity of TNF-α and ferroptosis-like cell death. The present invention also disclosed the preparation method and the use thereof in preparing a drug for treating the diseases mediated by TNF-α and/or iron-dependent cell death.

### FIELD OF INVENTION

The invention belongs to the technical field of medicine and provides a class of selenium-containing isoxazolamine derivatives as well as their pharmaceutically acceptable salts, solvates, polymorphs, stereoisomers, isotopic compounds, or metabolites, which can regulate the generation and/or activity of TNF-α and ferroptosis-like cell death. The invention also provides the preparation method and the use thereof for preventing and/or treating diseases associated with the TNF-α and/or ferroptosis pathway abnormalities in humans or other mammals.

### PRIOR ARTS

TNF-α (tumor necrosis factor-a): TNF-α is discovered in the 1970s, and recognized as a kind of proinflammatory cytokine which plays an important role in immune homeostasis, inflammation, and host defense. TNF-α is mainly secreted by activated monocytes, macrophages and T cells, and could activate the membrane receptors of caspase protease, JNK, and the transcription factor NF-κB, thus regulate a number of different biological processes, such as cell growth and apoptosis, tumor formation, immunity, inflammation and stress response, and so on. Moreover, TNF-α can also be secreted by cancer cells such as myeloid cells, which can promote tumor cells formation, angiogenesis, immune cell activation, differentiation, and cancer cell migration. Uncontrolled activity of TNF-α or overproduction of TNF-α is associated with the pathology of various diseases, including but not limited to cancers and inflammatory diseases, such as systemic inflammatory response syndrome, inflammatory bowel disease, rheumatoid arthritis, neurodegenerative diseases (multiple sclerosis, Motor neuron disease, Alzheimer's disease, Parkinson), psoriasis, cerebral malaria, diabetes, osteoporosis, allograft rejection, multiple sclerosis, HBV, HCV and HIV, etc. (Brenner D. et. al. Regulation of tumor necrosis factor signaling: live or let die. Nat Rev Immunol. 2015, 15 (6), 362; Blake Bartlett. et. al. Nat Rev Cancer. 2004, 4, 314.). Therefore, reducing the level of TNF-α, or regulating the activity of TNF-α is a promising strategy in treating many immunological, inflammatory, and malignant diseases (Front. Biosci. 2008, 13, 5094; Results Prob. Cell Differ. 2009, 49, 1).

So far, several TNF-α-targeted drugs have been developed, which contain bio-macromolecule drugs such as Infliximab, CD571, Etanercept, Onercept, Adalimmab (D2E7), CDP870, and small-molecule drugs such as Thalidomide, Pomadomide and Lenalidomide. These bio-macromolecule TNF-α inhibitors while showed obvious advantages of rapid and effective treatment for rheumatoid arthritis, mandatory spondylitis, dry moss arthritis, psoriasis, and inflammatory bowel disease, but also meet some disadvantages such as poor stability, poor tissue distribution, administration inconvenient, immune tolerance, and high cost. It is worthy of note that small molecule TNF-α inhibitors such as thalidomide and lenalidomide could overcome the disadvantages of bio-macromolecule drugs, and have been widely used in clinical for the treatment of erythematous nodular leprosy and malignant diseases such as myelodysplastic syndrome, myelofibrosis, mantle cell lymphoma, acute myeloid leukemia, and acute/chronic graft-versus-host response, ovarian cancer, renal cell carcinoma and other diseases (Palladino M A, et. al. Anti-TNF-α therapies: the next generation. Nat Rev Drug Discov. 2003, 2, 737). However, during the clinical trials, these small molecule TNF-α inhibitors showed poor long-term drug tolerance toxicities including peripheral neuropathy, drowsiness, constipation, and the risk of thromboembolism and teratogenicity greatly, which limited their potency and reduced the medication compliance of patients. Therefore, thalidomide derivatives being of improved structures are urgently desired to optimize its performance in the field.

Ferroptosis is a regulated form of cell death driven by loss of activity of the lipid repair enzyme glutathione peroxidase 4 (GPX4) and subsequent accumulation of lipid-based reactive oxygen species (ROS), particularly lipid hydroperoxides. This form of iron-dependent cell death is genetically, biochemically, and morphologically distinct from other cell death modalities, including apoptosis, unregulated necrosis, and necroptosis, which mainly shows increased cytoplasm and lipid active oxygen, smaller mitochondria, and higher mitochondrial membrane density. Ferroptosis is mainly regulated by intracellular signaling pathways, including iron homeostasis regulatory pathways, RAS pathways and cystine transport pathways, which are tightly involved in tumors, nervous system, coronary heart disease, tissue ischemia-reperfusion injury, acute renal failure, aging and other diseases. Glutathione peroxidase (GPX4) and thioredoxin reductase (TrxR) are two important seleno-proteinases the redox-system of the organism and play an important role in the death of iron in cells (DIXON, S. J. et al. Cell. 2012, 5, 1060; Ingold, I. et. al. Cell. 2018, 172, 409; Liabani, E. et al. Nat Chem. 2019, 11, 521). For example, loss of GPX4 activity and subsequent accumulation of lipid hydroperoxides executes normal cell ferroptosis. In addition, TrxR is a seleon-protein associated with various hematomas (such as lymphoma, multiple myeloma) and solid tumors (such as lung, liver, breast, and glioma), and plays an important role in the proliferation and differentiation of tumor cells. Consequently, the inhibition of TrxR could promote the ferroptosis process of cancer cell.

Basing on the above analysis of thalidomide drugs, a series of novel selenium-containing isoxazolamines were developed in this present invention. The results of pharmacodynamical researches showed that these selenium-containing isoxazolamines not only exhibiting moderate to good potency of TNF-α inhibition, but also could protect the ferroptosis of normal cells via GPX4 mimic, and promote the ferroptosis of tumor cells via TrxR inhibiting, thereby meeting the requirement of improving the therapeutic index of thalidomide derivatives.

### Content of the Present Invention

A general object of the present invention is to provide a series of novel selenium-containing isoxazolamine structure compounds.

A more specific object of the present invention is to provide a method for preparing these selenium-containing isoxazolamines.

Another object of the present invention is to provide the use of these selenium-containing isoxazolamines, which can regulate the production or activity of TNF-α and the ferroptosis process of cell lines, thus can be effectively used for treating cancers and inflammatory diseases.

The present invention provides a new type of selenium-containing isoxazolamine derivatives represented by general formula (I), a pharmaceutically acceptable salt, a solvate, a polymorph, a stereoisomer, an isotopic or a metabolite compound thereof;

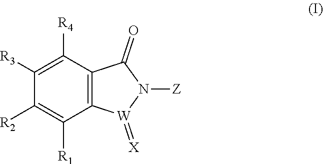

In the general formula (I),

each of R₁, R₂, R₃ and R₄ is independently selected from H, D, halogen, hydroxyl, amino, nitro, cyano, carboxyl, seleno, mercapto, (C₁-C₈) alkylselenyl, (C₁-C₈) alkylselenyl (C₁-C₈) alkylamino, (C₂-C₈) alkenylselenyl, α-(C₁-C₈) alkylselenyl amino acid, α-(C₁-C₈) alkylselenyl formyl amino acid, (C₀-C₈) alkylamino (C₁-C₈) alkylselenyl, (C₀-C₈) alkylaminoformylselenyl, (C₀-C₈) alkylaminoformyl, arylselenyl, (C₀-C₈) alkoxyl (C₁-C₈) alkylselenyl, (C₀-C₈) alkoxyformyl (C₁-C₈) alkylselenyl, (C₀-C₈) alkoxyformyl C₁-C₈ alkoxyl, halo (C₁-C₈) alkylselenyl, C₁-C₈ alkanesulfonyl, (C₁-C₈) alkanesulfonamido, (C₀-C₈) alkylaminosulfonyl, (C₁-C₈) alkyl, halo (C₁-C₈) alkyl, halo (C₁-C₈) alkoxyl, (C₀-C₈) alkylethynyl, (C₁-C₈) alkoxyl, (C₁-C₈) alkylacyloxy, (C₁-C₈) alkoxyl (C₁-C₈) alkoxyl, (C₁-C₈) alkoxyl (C₁-C₈) alkyl, (C₁-C₈) alkylamino, (C₀-C₈) alkylamino (C₁-C₈) alkyl, aryl, aryl (C₁-C₈) alkylamino (C₁-C₈) alkyl, amidino, guanidino, arylsulfonamido, arylaminosulfonyl, benzoyl, (C₀-C₈) alkylselenyl formyl, aryl (C₁-C₈) alkylamino, aryl (C₁-C₈) alkylamido, (C₁-C₈) alkoxyformyl, (C₁-C₈) alkylamido, (C₁-C₈) alkylamino, (C₀-C₈) alkylselenyl formamido, arylselenyl (C₁-C₈)alkylamido, selenylcyano (C₁-C₈) alkylamido, benzoisoselenidazolone amino, benzoisoselenidazolone amino (C₁-C₈) alkylamido, benzoisoselenidazolone amino (C₁-C₈) alkanesulfonamido, (C₀-C₈) alkylamino selenyl, (C₀-C₈) alkylaminoformyl, (C₀-C₈) alkylamino formylselenyl, (C₁-C₈) alkylaminoformyloxyl, (C₁-C₈) alkylaminoformyl, (C₁-C₈) alkylaminoformyloxy, arylaminoformamido, arylaminoformyl, arylaminoformyloxy, piperidinyl, piperazinyl, morpholinyl, pyrrolyl, pyrazolyl, imidazolyl, pyridyl, pyrazinyl, quinolinyl, pyrimidinyl, pyrimidinylamino, thiazolyl, thienyl, furanyl, pyrrolyl or absent; wherein, the aryl groups of R₁, R₂, R₃ and R₄ described are phenyl or are phenyl which independently substituted with 1-4 halogen, hydroxy, nitro, cyano, amino, trifluoromethyl, carboxyl, (C₀-C₈) alkylaminosulfonyl, (C₁-C₈) alkanesulfonamido, (C₁-C₈) alkyl, halo (C₁-C₈) alkoxyl, (C₁-C₈) alkoxyl groups;

Z is:

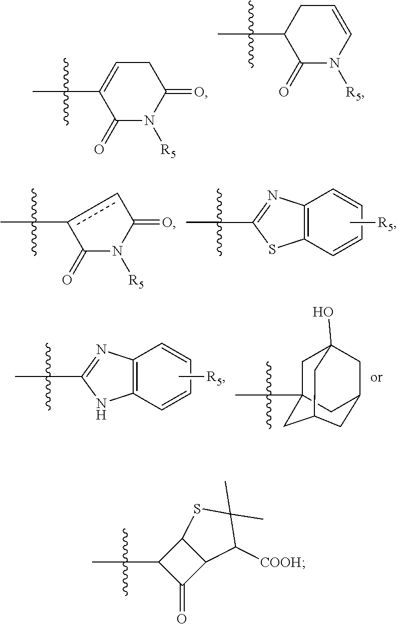

wherein Z is

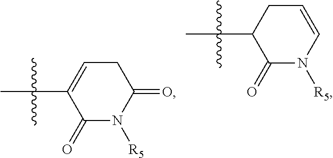

R₅ is selected from H, D, (C₁-C₈) alkylselenyl (C₁-C₈) alkyl, (C₂-C₈) alkenylselenyl (C₁-C₈) alkyl, selenocyanate (C₁-C₈) alkyl,

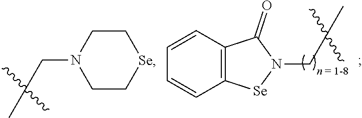

wherein Z is

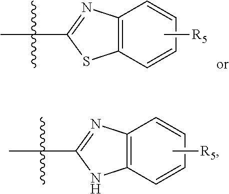

R₅ is is selected from H, halogen, hydroxyl, nitro, cyano, amino, trifluoromethyl, carboxyl, (C₁-C₈) alkanesulfonyl, amino sulfonyl, (C₁-C₈) alkyl, halo (C₁-C₈) alkoxyl, (C₁-C₈) alkoxyl;

W is C or Se; wherein, when W is C, there is one selenium-containing substituent exists in the R₁, R₂, R₃, R₄, and R₅ group at least; and when W is Se, R₁, R₂, R₃, R₄, and R₅ could be any substitutes as described above;

X is, O or not exist;

Where bonds represented by “ [symbol] ” is chemical bond or not exist.

Preferably, the present invention provides a series of compounds represented by formula (I-a), (I-b), (I-c), (I-d), and (I-e), as well as a pharmaceutically acceptable salt, a solvate, a polymorph, a stereoisomer, an isotopic or a metabolite compound thereof;

Preferably, the present invention provides a series of compounds represented by formula (I-a), (I-b), (I-c), (I-d), and (I-e), as well as a pharmaceutically acceptable salt, a solvate, a polymorph, a stereoisomer, an isotopic or a metabolite compound thereof:

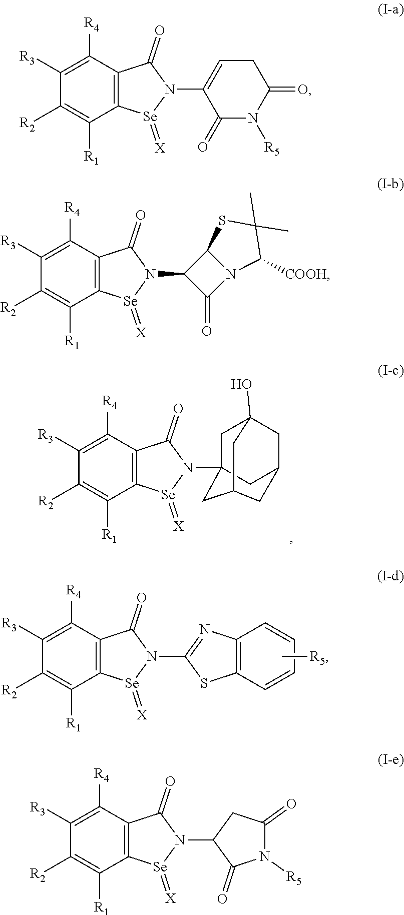

In the general formula (I-a˜I-e), each of R₁, R₂, R₃ and R₄ is independently selected from H, D, halogen, hydroxyl, amino, nitro, cyano, carboxyl, (C₀-C₈) alkylamino (C₁-C₈) alkylselenyl, (C₀-C₈) alkylaminoformyl (C₁-C₈) alkoxyl, amidino, guanidino, C₁-C₈ alkanesulfonyl, (C₁-C₈) alkanesulfonamido, (C₀-C₈) alkylaminosulfonyl, (C₁-C₈) alkyl, halo (C₁-C₈) alkyl, halo (C₁-C₈) alkoxyl, (C₀-C₈) alkylethynyl, (C₁-C₈) alkoxyl, (C₁-C₈) alkylacyloxy, (C₁-C₈) alkoxyl (C₁-C₈) alkoxyl, (C₁-C₈) alkoxyl (C₁-C₈) alkyl, (C₁-C₈) alkylamino, (C₀-C₈) alkylamino (C₁-C₈) alkyl, aryl, aryl (C₁-C₈) alkylamino (C₁-C₈) alkyl, arylsulfonamido, arylaminosulfonyl, benzoyl, arylmethylamino, aryl formamido, (C₀-C₈) alkoxyformyl, (C₁-C₈) alkylamido, (C₁-C₈) alkylamino, (C₀-C₈) alkylaminoformamido, (C₀-C₈) alkylaminoformyl, arylaminoformamido, arylaminoformyloxy or absent; wherein, the aryl groups of R₁, R₂, R₃ and R₄ described are phenyl or are phenyl which independently substituted with 1-4 halogen, hydroxy, nitro, cyano, trifluoromethyl, carboxyl, aminosulfonyl, (C₁-C₆) alkyl, (C₁-C₆) alkoxyl groups;

where Z is selected from

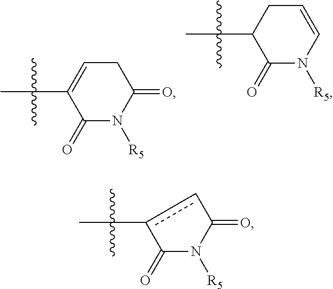

R₅ is H, D, (C₁-C₈) alkylselenyl (C₁-C₈) alkyl; where Z is selected from

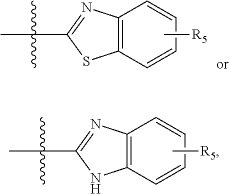

R5 is H, halogen, hydroxyl, nitro, cyano, amino, trifluoromethyl, carboxyl, (C₁-C₈) alkanesulfonyl, aminosulfonyl, (C₁-C₈) alkyl, halo (C₁-C₈) alkoxyl, (C₁-C₈) alkoxyl;

X is, O or not exist;

where bonds represented by “ [symbol] ” is chemical bond or not exist.

As used herein, the term “halogen” refers to fluorine, chlorine, bromine, and iodine.

As used herein, the term “halogenated” may have either a mono halogenated, or a poly halogenated.

As used herein, the term “alkanesulfonyl” refers to a linear or branched or cyclic saturated alkylsulfonyl group, and the cyclic saturated alkane described contains 3 to 8 carbon atoms.

As used herein, the term “alkanesulfonamido” refers to a linear or branched or cyclic saturated alkylsulfonamide group, and the cyclic saturated alkane described contains 3 to 8 carbon atoms.

As used herein, the term “alkylaminosulfonyl” refers to a N-monosubstituted or disubstituted linear or branched or cyclic saturated alkane aminosulfonyl group, and the cyclic saturated alkane described contains 3 to 8 carbon atoms.

As used herein, the term “alkylaminoformyl” refers to a N-monosubstituted or disubstituted linear or branched or cyclic saturated alkane aminoformyl group, and the cyclic saturated alkane described contains 3 to 8 carbon atoms.

As used herein, the term “alkane” refers to a linear or branched or cyclic saturated alkyl group, and the cyclic saturated alkane described contains 3 to 8 carbon atoms.

As used herein, the term “alkoxy” refers to a linear or branched or cyclic saturated alkoxyl group, and the cyclic saturated alkane described contains 3 to 8 carbon atoms.

As used herein, the term “alkylethynyl” refers to a linear or branched or cyclic saturated alkane ethynyl, and the cyclic saturated alkane described contains 3 to 8 carbon atoms.

As used herein, the term “alkylacyloxy” refers to a linear or branched or cyclic saturated alkane acyloxy group, and the cyclic saturated alkane described contains 3 to 8 carbon atoms.

As used herein, the term “alkylamino” refers to a N-monosubstituted or disubstituted linear or branched or cyclic saturated alkane amine, and the cyclic saturated alkane described contains 3 to 8 carbon atoms.

As used herein, the term “alkoxyformyl” refers to a linear or branched or cyclic saturated alkoxyformyl group, and the cyclic saturated alkane described contains 3 to 8 carbon atoms.

As used herein, the term “alkylamido” refers to a linear or branched or cyclic saturated alkane amido group, and the cyclic saturated alkane described contains 3 to 8 carbon atoms.

As used herein, the term “alkylaminoformamido” refers to a linear or branched or cyclic saturated alkane aminoformamido group, and the cyclic saturated alkane described contains 3 to 8 carbon atoms.

As used herein, the term “stereoisomers” refers to the chiral compounds that contain one or more stereocenters, the term “stereoisomer” herein including enantiomer, diastereoisomer.

As used herein, unless otherwise specified, the substituted attachment site of “piperazinyl, morpholinyl, pyrrolyl, pyrazolyl, pyrrolyl, imidazolyl, pyrimidylamino” groups are at the nitrogen atom.

As used herein, unless otherwise specified, the substituted attachment site of “pyridyl, pyrimidinyl, thiazolyl, thienyl, furanyl, pyrazinyl, quinolinyl” are at the carbon atom.

In the case where the compounds described in the present invention have stereoisomers, the stereoisomers include all stereoisomers of the compounds.

The present invention also includes deuterated compounds which refers to one or more of the hydrogen atoms in the compound is replaced by its heavier isotope deuterium.

As used herein, the term “metabolite” refers to an active substance produced after the chemical structure of a drug molecule changes in vivo, the active substance is generally a derivative of the aforementioned drug molecule, and also can be chemically modified.

As used herein and unless otherwise specified, the term“polymorph” refers to one or more than one kind(s) of crystal structure formed by the different arrangement of molecules in the lattice space when crystallizing.

As used herein, the term “solvate” refers to a crystal form of the compound having a structure of general formula (I), the pharmaceutically acceptable salt, the polymorph, the stereoisomer, the isotopic compound, the metabolite or the prodrug thereof, which further has one or more than one kind(s) of solvent molecule(s) incorporated into the crystal structure. The solvate may include a stoichiometric amount or a non stoichiometric amount of solvent, and the solvent molecule in the solvent may exist in an ordered or non ordered arrangement. The solvate containing a non stoichiometric amount of solvent molecules may be formed by losing at least one solvent molecule (but not all) from the solvate. In a particular embodiment, a solvate refers to a hydrate, which means the crystal of the compound further includes water molecule, and water is used as a solvent.

The compound having a structure of general formula (I) in the present invention, the pharmaceutically acceptable salt, the solvate, the polymorph, the stereoisomer, the isotopic compound or the metabolite thereof, can contain one or more than one asymmetric centers (“stereoisomer”). As used herein, the term “stereoisomer” refers to all stereoisomers including enantiomer, diastereoisomer, epimer, endo-exo isomer, atropisomer, regioisomer, cis- and trans-isomer. The “stereoisomer” herein also includes “pure stereoisomer” and “enriched stereoisomer” or “racemic isomer” of the various aforementioned stereoisomers. These stereoisomers can be prepared according to an asymmetric synthesis process, or separated, purified, and enriched by a chiral separation process (including but not limited to thin layer chromatography, rotating chromatography, column chromatography, gas chromatography, high pressure liquid chromatography, etc.), as well as obtained by chiral separation by means of bonding (chemical binding etc.) or salifying (physical binding etc.) with other chiral compound(s).

The compounds of general formula (I), pharmaceutically acceptable salts, solvates, crystalline forms, stereoisomers, isotopic compounds, or metabolites of the invention may contain one or more asymmetric centers (“stereoisomers”). As used herein, the term “stereoisomers” refers to enantiomers, diastereomers, epimers, endo-exo isomers, atropis All stereoisomers including atropisomers, regioisomers, cis- and trans-isomers, etc. The “stereoisomers” herein also include “pure stereoisomers” and “enriched stereoisomers” or “racemates” of the aforementioned various stereoisomers. These stereoisomers can be separated, purified, and enriched by asymmetric synthesis methods or chiral separation methods (including but not limited to thin-layer chromatography, rotary chromatography, column chromatography, gas chromatography, high-pressure liquid chromatography, etc.), and can also be purified by It can be obtained by chiral resolution by bonding with other chiral compounds (chemical bonding, etc.) or salt formation (physical bonding, etc.).

As used herein, the term “pharmaceutically acceptable salt” refers to a non-toxic acid salt of the compounds of formula I. These salts can be prepared in situ during the final isolation and purification of compounds of general formula I, or can be synthesized by reacting appropriate organic or inorganic acids with basic functional groups, respectively. Examples of the salt include but are not limited to, acetate, adipate, alginate, citrate, aspartate, benzoate, benzenesulfonate, hydrogen sulfate, butyrate, Camphor salt, camphor sulfonate, digluconate, cyclopentanepropionate, dodecyl sulfate, ethanesulfonate, glucoheptanoate, glyceryl phosphate, hemisulfate, heptanoate Salt, hexanoate, fumarate, hydrochloride, hydrobromide, hydroiodate, 2-hydroxyethanesulfonate, lactate, maleate, mesylate, nicotinate, 2-naphthylsulfonate, oxalate, paraben, pectate, thiocyanate, 3-phenylpropionate, picrate, pivalate, propionate, amber Acid salt, sulfate, tartrate, thiocyanate, p-toluene sulfonate and undecanoate.

Some of the preferred novel selenium-containing isoxazolium compounds of the present invention are shown below. These examples are used for further explanation of the present invention only, and do not limit the scope of the present invention in any way.

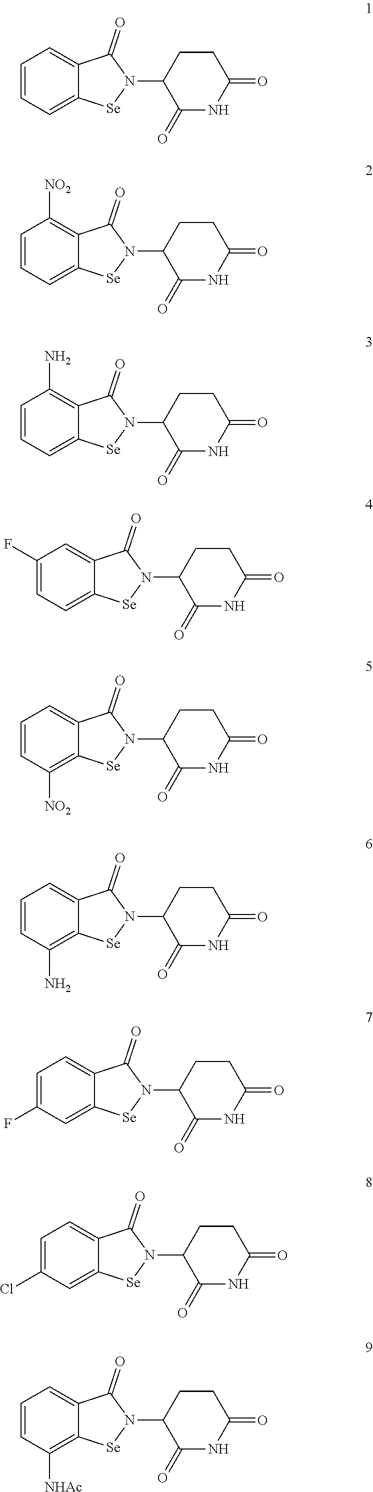

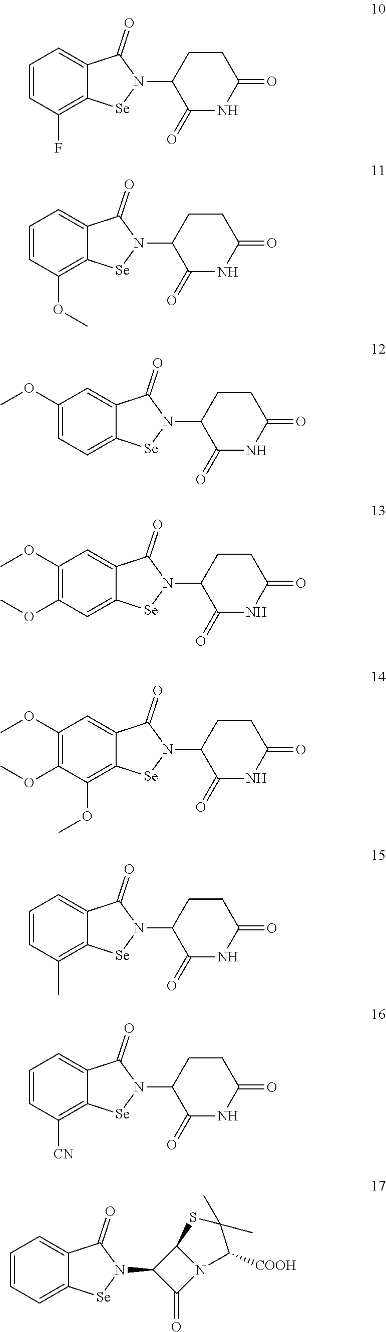

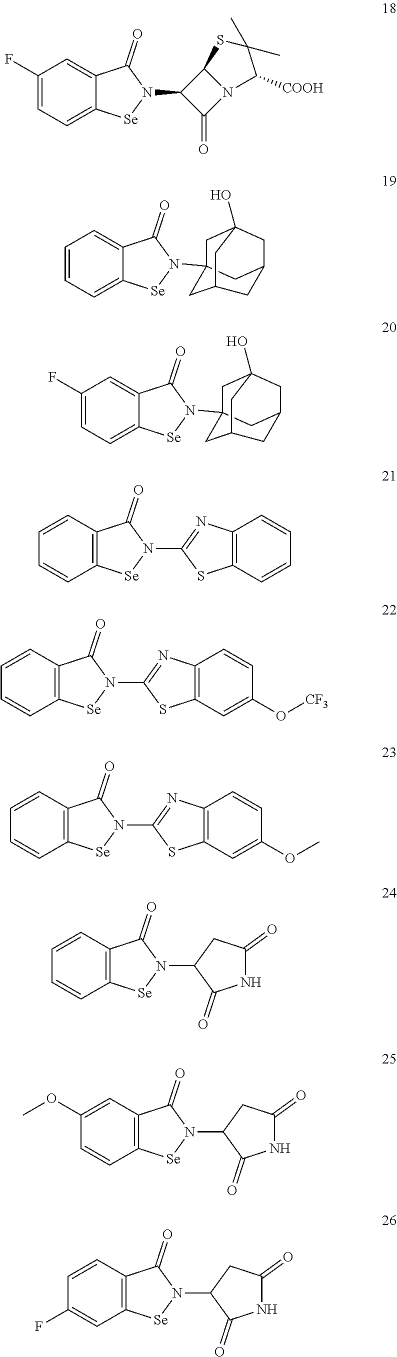

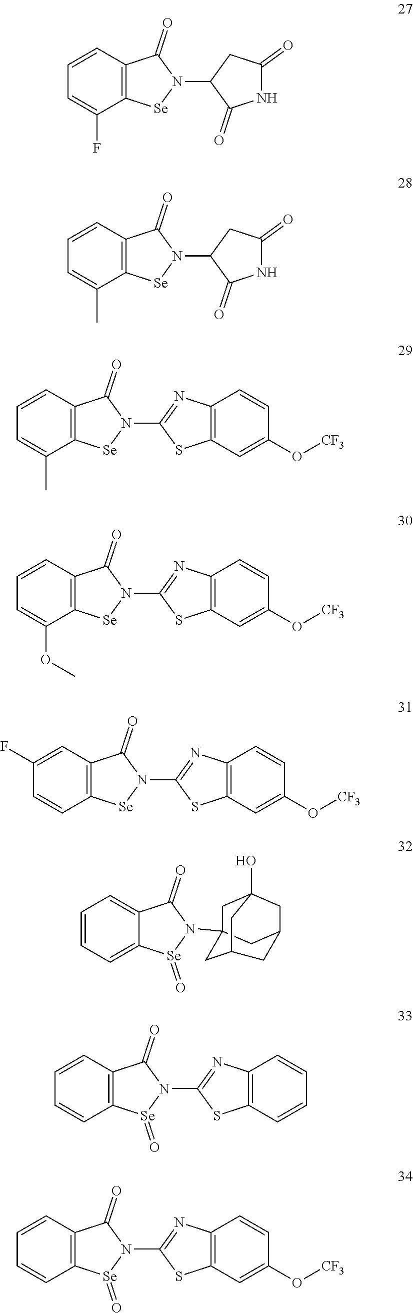

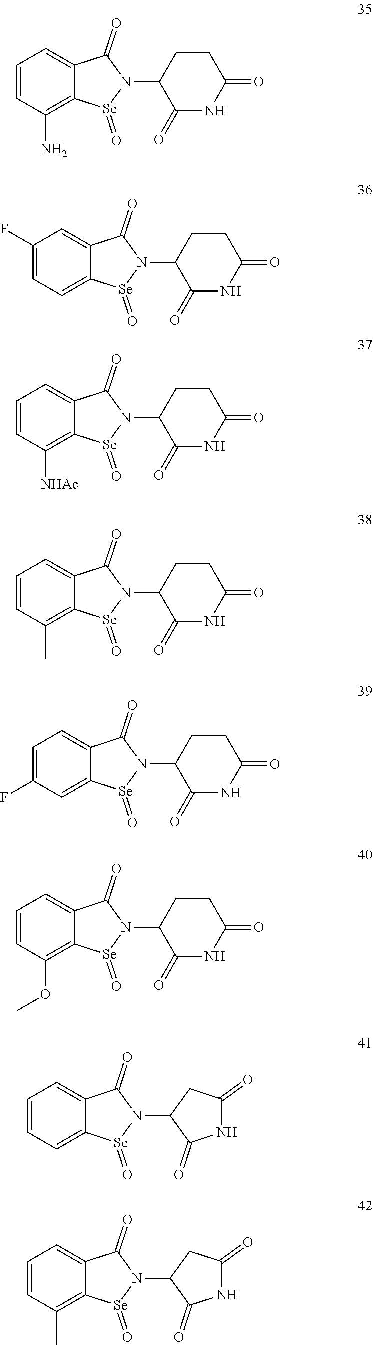

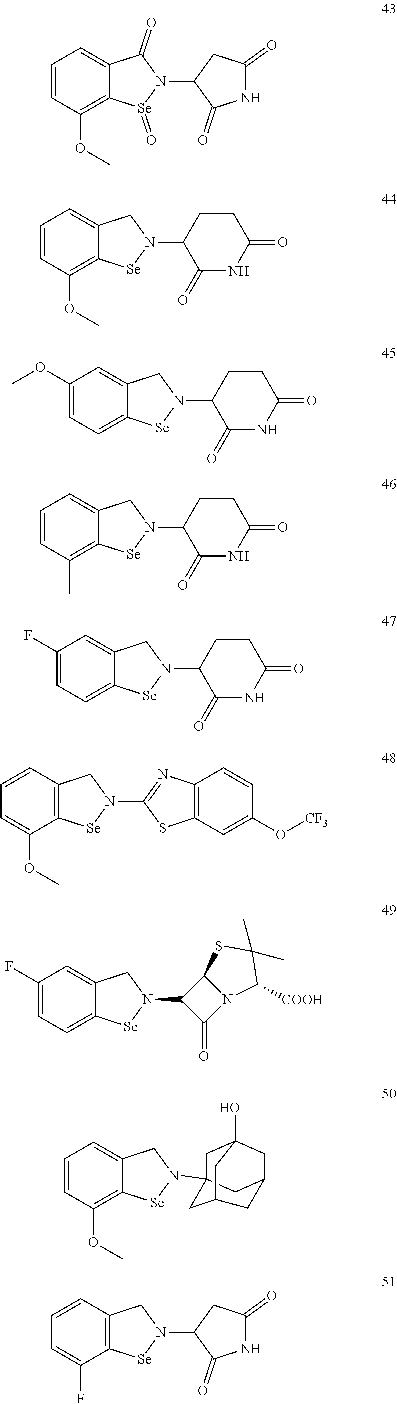

As is known to all, any stereocenter of the above-listed compounds that is not explicitly stated can be an absolute (R)- or (S)-configuration, or its racemic mixture. The present invention includes the racemic mixtures of the compounds, a mixture of any one of enrichment enantiomer, and any one of isolated enantiomer. For the scope of the present invention, the racemic mixture refers to a 50%: 50% mixture of R and S enantiomer, and the isolated enantiomer should be understood as pure enantiomer (such as 100%) or a highly enriched mixture of certain enantiomer (purity ≥98%, ≥95%, ≥90%, ≥88%, ≥85%, ≥80%).

The present invention also provides a pharmaceutically acceptable salt of the above-mentioned novel selenium-containing isoxazolamines.

According to the second object of the present invention, the methods for the preparation of selenium-containing isoxazolamines and/or their pharmaceutically acceptable salts are provided as follows.

The following abbreviations shall apply throughout the specification and examples:

Ac refers to acetyl group. AcOH refers to acetic acid. Base refers to organic or inorganic base. DMF refers to N,N-dimethylformamide. EA refers to ethyl acetate. EtOH refers to ethanol. EDC refers to 1-Ethyl-(3-dimethylaminopropyl) carbodiimide hydrochloride. HA refers to organic or inorganic acids, such as hydrochloric acid, sulfuric acid, maleic acid, tartaric acid, etc. H₂O₂ refers to hydrogen peroxide. HOBt refers to 1-hydroxybenzotriazole. OMs refers to methanesulfonyloxy. LC-MS refers to high performance liquid chromatography-mass spectroscopy. NMR refers to nuclear magnetic resonance chromatograph. Pd/C H₂ refers to palladium carbon hydrogen reduction system. TLC refers to thin layer chromatography. V refers to solution volume.

The present compound of formula I can be prepared according to the following general method:
(1) The first synthesis route for preparing some of the selenium-containing isoxazolamines represented by general formula I-a, I-b, I-c, I-d and I-e.

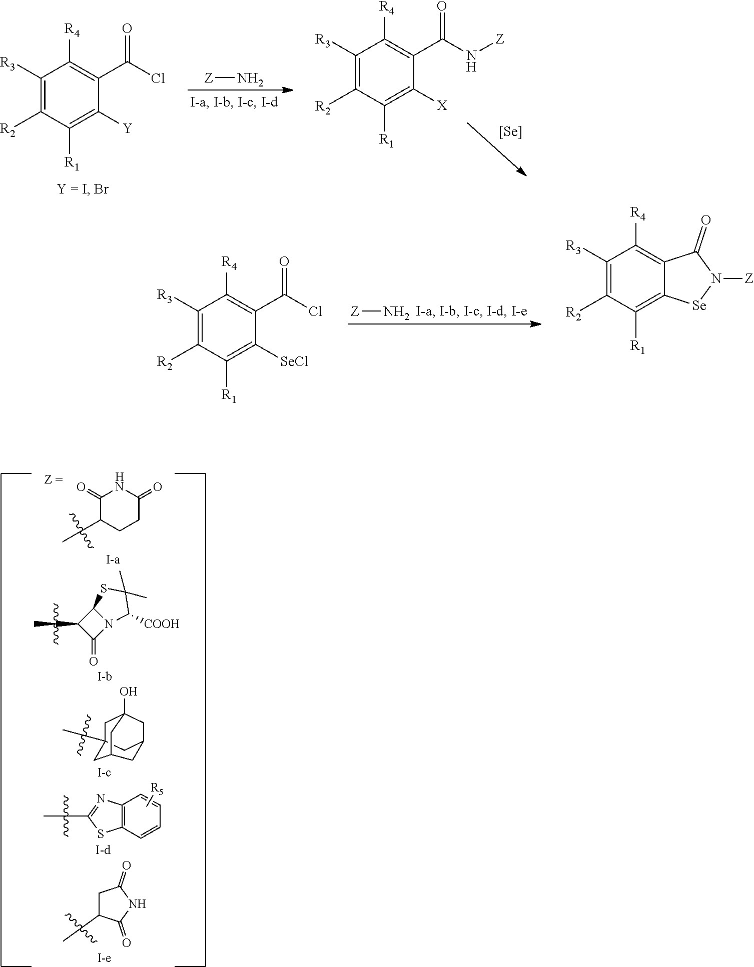

(2) The second synthesis route for preparing some of the selenium-containing isoxazolamines represented by general formula I-a, I-b, I-c, I-d, and I-e.

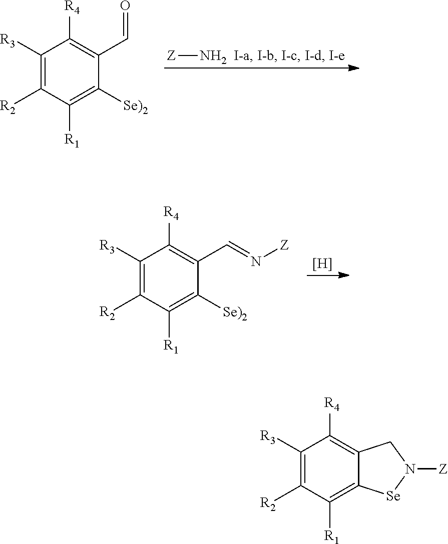

(3) The third synthesis route for preparing tetravalent selenium compounds represented by general formula I-a, I-b, I-c, I-d and I-e.

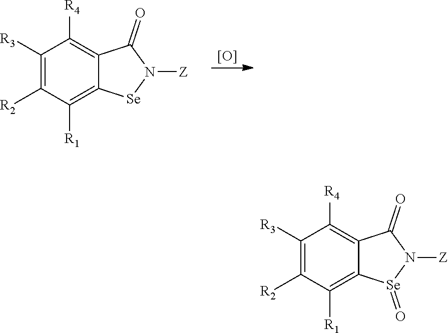

In the first synthesis route for preparing the selenium-containing isoxazolamines represented by general formula I-a, I-b, I-c, I-d and I-e, the different substituted benzoyl chloride can react with different 3-amino-2,6-piperidinedione or 3-amino-1,4-dihydropyridine-2-(1H)-one or 3-amino-1-adamantanol or 2-benzothiazolamine or 3-amino-2,5-pyrroledione respectively to afford the corresponding desired products in the presence of tertiary amines such as triethylamine, diisopropylethylamine under heated condition (rt˜120° C.), among which, the solvents used include, but is not limited to, N,N-dimethylformamide, dimethylsulfoxide, acetonitrile, tetrahydrofuran, dichloromethane, chloroform, ethyl acetate, and so on (References: Heteroatom Chemistry. 2014, 35, 320). Wherein, when the ortho substituent of benzoyl chloride is Y=SeCl, the desired benzoisoselazolidone derivatives of formulas I-a, I-b and I-e can be directly obtained by reacting with the above substrates (References: J. Med. Chem. 2013, 56, 9089); when the ortho substituent of benzoyl chloride is Y=I or Br, the reaction with the above substrates will first obtain the corresponding o-halides benzamide intermediates respectively, and then can produce the desired benzoisoselazolidone derivatives of formulas I-a, I-b, I-c and I-e via the [Se] reaction (References: Org. Lett. 2010, 12, 23; J. Org. Chem. 2017, 82, 3844; Tetrahedron. 2011, 67, 9 565).

In the second synthesis route for preparing the selenium-containing isoxazolamines represented by general formula I-a, I-b, I-c, I-d, and I-ef, the different substituted 2,2′-diselenylbisbenzaldehydes can respectively react with different 3-amino-2,6-piperidinedione or 3-amino-1,4-dihydropyridine-2-(1H)-one or 3-amino-1-adamantanol or 2-benzothiazolamine or 3-amino-2,5-pyrroledione to afford the corresponding imine intermediates, which can produce the desired selenium-containing isoxazolamines via the reductive amination (Reference: Angew. Chem. Int. Ed. 2015, 54, 1).

In the third synthesis route for preparing the tetravalent selenium-containing isoxazolamines represented by general formula I-a, I-b, I-c, I-d, and I-e, the benzisoselenidazole derivatives can be direct oxidated to the desired products with [O⁻] reagents, and the solvent includes, but not limited to, tetrahydrofuran, dichloromethane, chloroform, and ethyl acetate, and the reaction temperature is −20° C. to 0° C. The [O⁻] peroxide reagents used includes, but is not limited to, H₂O₂, O₃, and m-chloroperoxybenzoic acid (Reference: J. Org. Chem. 2005, 70, 868; J. Org. Chem. 2005, 70, 5023).

The derivatives of formula I-a, I-b, I-c, I-d, and I-e can be obtained by conventional post-treatment, the reaction process is usually monitored by TLC and LC-MS, after the reaction is co completed, extraction with a solvent such as methyl tert-butyl ether, ethyl acetate or dichloromethane, washing with saturated sodium bicarbonate, water and saturated brine in order, drying over anhydrous sodium sulfate or magnesium sulfate, and removing the solvent under reduced pressure at low temperature. The key intermediate and final products were confirmed by NMR and mass spectrometry.

The derivatives of the formula I-a, I-b, I-c, I-d and I-e that contain —NH₂, alkylamine or arylamine groups can combine with HA to produce the corresponding pharmaceutically acceptable salts. HA refers to hydrochloric acid, hydrobromic acid, sulfuric acid, phosphoric acid, acetic acid, citric acid, oxalic acid, malonic acid, salicylic acid, malic acid, fumaric acid, succinic acid, mandelic acid, ascorbic acid, maleic acid, tartaric acid, benzenesulfonic acid, methanesulfonic acid or isethionate.

According to the third object of the present invention, the derivatives of the formula I can inhibit the overexpression of TNF-α and normal cells ferroptosis. Accordingly, they can be used as TNF-α inhibitor and/or ferroptosis inhibitor for the treatment (including combination therapy) of the diseases related to TNF-α overexpression and/or ferroptosis-like cell death, such as autoimmune diseases, hematological tumors, solid tumors, tissue ischemia-reperfusion injury, acute renal failure, and aging diseases. The autoimmune diseases include myelofibrosis, acute/chronic graft-versus-host response disease, rheumatoid arthritis, inflammatory bowel disease, diabetes, psoriasis, mandatory spondylitis, leprosy nodular erythema, and other infectious diseases such as HBV, HCV, HIV; the neurodegenerative diseases include Alzheimer's disease, dementia, multiple sclerosis, motor neuron disease; the blood tumor refers to multiple bone marrow tumor, myelodysplastic syndrome; the solid tumor refers to liver cancer, kidney cancer, gastric cancer, colon cancer, ovarian cancer, pancreatic cancer, prostate cancer, breast cancer, melanoma, and cerebral glioblastoma; the tissue ischemic reperfusion injury refers to stroke, coronary heart disease, myocardial infarction, pulmonary embolism, and acute coronary syndrome.

### Beneficial Effect

The present invention developed a new series of selenium-containing isoxazolamines, which not only showed a significant inhibitory effect on TNF-α, but also could mimic the selenium enzymes of GPX4 to regulate the ferroptosis-like cell death comparing with the known thalidomides, thereby meeting the therapeutic requirement of complex diseases such as neurodegenerative diseases and autoimmune diseases.

### BRIEF DESCRIPTION OF THE DRAWINGS

FIG. 1 is the general structure of. the compounds of the present invention according to formula I.

FIG. 2 is the protective effect of the compound of formula I on ox-LDL-induced human vascular endothelial cell injury.

FIG. 3 is the protective effect of the compound of formula I on erastin-induced ferroptosis-like cell death in HT22 cells.

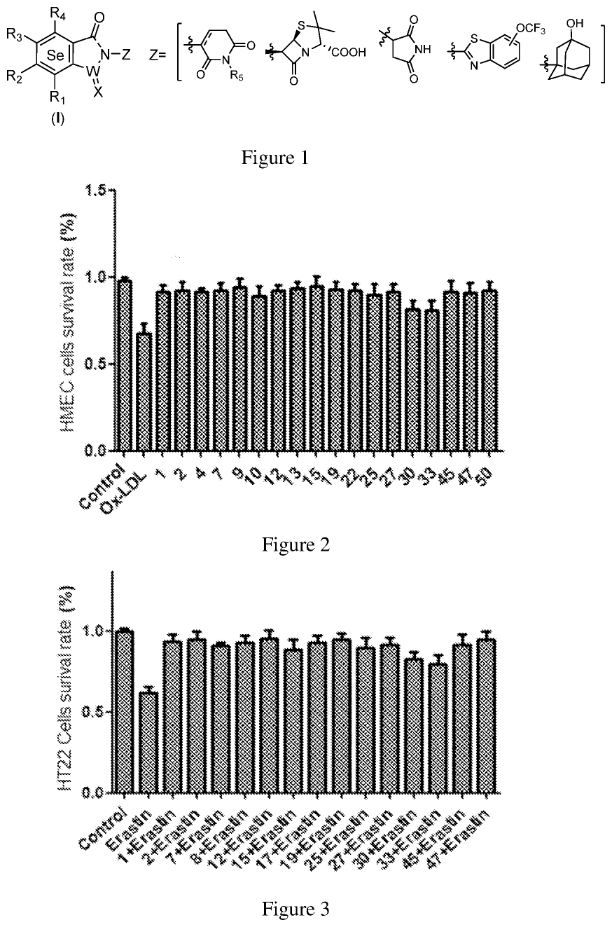

### EXAMPLES

The present invention will now be further elucidated by way of a description of a preferred exemplary embodiment of the invention, but is not limited thereto. Each preferred conditions aforementioned can be combined randomly without departing from the common knowledge in the art thereby forming various preferred embodiments of the present invention.

In the following embodiments, 1H-NMR was measured with a Varian Mercury AMX300 instrument. MS was measured with VG ZAB-HS or VG-7070 and Esquire 3000Plus-01005. All reaction solvents are redistilled before use, and the anhydrous solvents are obtained in accordance with standard drying methods. Unless otherwise indicated, all reactions were carried out under the protection of argon and monitored by TLC, and following the conventional workup and pre-drying treatment by saturated saline and anhydrous sodium sulfate. Products were purified by column chromatography on silica gel (200-300 mesh) unless otherwise stated.

### Example 1. Synthesis of Compound 1

Step 1: Preparation of 2-Chloroselenobenzoyl Chloride

Anthranilic acid (1.37 g, 10 mmol) was added to a 3N aqueous hydrochloric acid solution (4 ml) under an ice bath, and then sodium nitrite (10 mmol, 690 mg) in an aqueous solution (2 ml) was slowly added to the reaction mixture under stirring, and the reaction will change to a clear solution after an hour and to give the desired 2-benzoic acid diazonium salt solution.

Selenium powder (790 mg, 10 mmol) was mixed with cetyltrimethyl ammonium bromide (20 mg) and 2N aqueous sodium hydroxide solution (5 ml) to obtain a Se—NaOH solution under nitrogen atmosphere. NaBH₄ (49 mg, 1.3 mmol) solution (1 mL) that contained NaOH (40 mg, 1 mmol) was added drop-wisely into the Se—NaOH solution under an ice bath. The reaction was stirred at room temperature for 1 h and then at 90° C. for 0.5 h to from Na₂Se₂. After cooling to the room temperature, the 2-benzoic acid diazonium salt solution obtained above was slowly added dropwise to the Na₂Se₂ solution, and the mixture was heated to 40° C. for 2 hours. After the reaction was completed, the reaction solution was filtered, and the filtrate was acidified by adding 6N HCl until the precipitate was no longer precipitated, filtered, the filter cake was washed with water and dried over to give a khaki solid of 2,2′-disselenized bisbenzoic acid in 80% yield, mp 295-296° C.

2,2′-Diselenized bisbenzoic acid (800 mg, 2 mmol) was added to a sulfoxide solution (5 ml), and the mixture was heated to reflux for 3 hours under nitrogen atmosphere. The excessive sulfoxide was removed by evaporation under vacuum, while the residue was extracted with anhydrous n-hexane. The combined organic phases were evaporated under vacuum to give a yellow solid, which could be recrystallized from diethyl ether to obtain 2-chloroselenobenzoyl chloride as a pale-yellow solid with a yield of 81%, mp 60-62° C.

Step 2: Synthesis of Target Compound 1

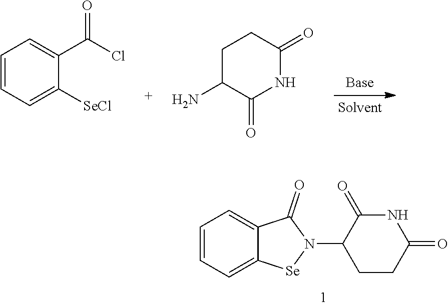

A solution of dichloroselenobenzoyl chloride (254 mg, 1 mmol) in acetonitrile (2 mL) was added dropwise to a stirred acetonitrile solution (10 mL) of 3-amino-2,6-piperidinedione (128 mg, 1 mmol) and triethylamine (151 mg, 1.5 mmol) under nitrogen atmosphere and ice bath (J. Med. Chem. 2016, 59, 8125-8133). After the reaction was completed (monitored by TLC), 20 ml of water was added, followed by extraction with ethyl acetate (20 mL×2). The organic phase was sequentially washed with saturated brine, dried over anhydrous sodium sulfate, and filtered. The resulting filtrate was evaporated under reduced pressure and purified by silica gel column chromatography. (V_{acetone}:V_{petroleum ether}=1:4 to 1:1) to give compound 1 (260 mg, yield 85%). HRMS-ESI: m/z calcd for C₁₂H₁₀N₂O₃Se: 309.9857, found [M+H]⁺ 310.9927; ¹H NMR (400 MHz, DMSO-d₆) δ 10.98 (s, 1H), 8.07 (d, J=8.0 Hz, 1H), 7.85 (d, J=7.4 Hz, 1H), 7.68-7.62 (m, 1H), 7.45 (t, J=7.4 Hz, 1H), 5.26 (dd, J=12.8, 5.3 Hz, 1H), 2.92-2.83 (m, 1H), 2.59 (d, J=17.3 Hz, 1H), 2.48-2.36 (m, 1H), 2.13-2.05 (m, 1H); ¹³C NMR (126 MHz, DMSO) δ 173.2, 171.3, 167.5, 140.4, 132.25, 128.0, 127.9, 126.3, 126.2, 53.7, 31.7, 25.1.

Example 2 to 14 were performed according to the operation of example 1, wherein the synthesis of substituted 2-chloroselenobenzoyl chloride could follow the reference methods (J. Med. Chem. 2016, 59, 8125-8133 or Bioorg Med Chem. 2012, 20, 3816-3827) that using 2-aminobenzoic acid as the raw material (commercially available) and following by diselenyl etherification and chlorination reaction. The compounds listed in examples 15 to 16 were synthesized according to the above route and using 6-aminopenicilanic acid instead of 3-amino-2,6-piperidinedione; the compounds listed in examples 17 to 21 were synthesized according to the above route and using 3-amino-2,5-pyrroledione instead of 3-amino-2,6-piperidinedione. Examples of results obtained are as follows:

| Examples | Compound information |
|---|---|
| 2 | Compound 2, molecular formula: C₁₂H₉N₃O₅Se, yield 48%. HRMS-ESI: |
| | m/z 354.9707, found [M + H]⁺ 355.9781. |
| 3 | Compound 4, molecular formula: C₁₂H₉FN₂O₃Se, yiled 72%. HRMS-ESI: |
| | m/z 327.9762, found [M + H]⁺ 328.9835; ¹H NMR (400 MHz, DMSO-d₆) δ |
| | 11.02 (s, 1H), 8.02-7.95 (m, 1H), 7.58-7.47 (m, 2H), 5.32-5.23 (m, |
| | 1H), 2.96-2.87 (m, 1H), 2.69-2.56 (m, 1H), 2.49-2.36 (m, 1H), 2.15- |
| | 2.03 (m, 1H). |
| 4 | Compound 5, molecular formula: C₁₂H₉N₃O₅Se, yiled 75%. HRMS-ESI: |
| | m/z 354.9707, found [M + H]⁺ 355.9785; ¹H NMR (400 MHz, DMSO-d₆) δ |
| | 11.11 (s, 1H), 8.57-8.40 (m, 2H), 7.87-7.70 (m, 1H), 5.32-5.23 (m, |
| | 1H), 2.96-2.87 (m, 1H), 2.68-2.58 (m, 1H), 2.49-2.37 (m, 1H), 2.15- |
| | 2.03 (m, 1H). |
| 5 | Compound 7, molecular formula: C₁₂H₉FN₂O₃Se, yiled 73%. HRMS-ESI: |
| | m/z 327.9762, found [M + H]⁺ 328.9837; ¹H NMR (400 MHz, DMSO-d₆) δ |
| | 11.01 (s, 1H), 8.04 (dd, J = 8.6, 5.3 Hz, 1H), 7.30-7.27 (m, 1H), 7.17 (dt, |
| | 8.6, 2.4 Hz, 1H), 5.30-5.23 (m, 1H), 2.96-2.87 (m, 1H), 2.64-2.55 (m, |
| | 1H), 2.49-2.36 (m, 1H), 2.17-2.02 (m, 1H). |
| 6 | Compound 8, molecular formula: C₁₂H₉ClN₂O₃Se, yiled 62%. HRMS-ESI: |
| | m/z 343.9467, found [M + H]⁺ 344.9540; ¹H NMR (400 MHz, DMSO-d₆) δ |
| | 11.03 (s, 1H), 8.01 (dd, J = 8.2, 3.5 Hz, 1H), 7.62-7.38 (m, 2H), 5.30-5.24 |
| | (m, 1H), 2.97-2.88 (m, 1H), 2.66-2.53 (m, 1H), 2.47-2.36 (m, 1H), |
| | 2.17-2.02 (m, 1H). |
| 7 | Compound 9, molecular formula: C₁₄H₁₃N₃O₄Se, yiled 58%. MS-ESI |
| | [M + H]⁺ 368.1. |
| 8 | Compound 10, molecular formula: C₁₂H₉FN₂O₃Se, yiled 52%. MS-ESI |
| | [M + H]⁺ 329.0. |
| 9 | Compound 11, molecular formula: C₁₃H₁₂N₂O₄Se, yiled 79%. HRMS-ESI: |
| | m/z 339.9962, found [M + H]⁺ 341.0035; ¹H NMR (400 MHz, DMSO-d₆) δ |
| | 10.81 (s, 1H), 7.95 (d, J = 7.8 Hz, 1H), 7.31 (d, J = 7.5 Hz, 1H), 7.20 (t, J = |
| | 7.8 Hz, 1H), 5.24-4.73 (m, 1H), 3.92 (s, 3H), 2.95-2.83 (m, 1H), 2.65- |
| | 2.53 (m, 1H), 2.48-2.32 (m, 1H), 2.12-1.95 (m, 1H). |
| 10 | Compound 12, molecular formula: C₁₃H₁₂N₂O₄Se, yiled 67%. HRMS-ESI: |
| | m/z 339.9962, found [M + H]⁺ 341.0033; ¹H NMR (400 MHz, DMSO-d₆) δ |
| | 10.95 (s, 1H), 7.85 (d, J = 8.8 Hz, 1H), 7.29-7.20 (m, 2H), 5.21 (dd, J = |
| | 12.8, 5.3 Hz, 1H), 3.82 (s, 3H), 2.92-2.83 (m, 1H), 2.59 (d, J = 17.3 Hz, |
| | 1H), 2.48-2.37 (m, 1H), 2.12-2.02 (m, 1H). |
| 11 | Compound 13, molecular formula: C₁₄H₁₄N₂O₅Se, yiled 83%. HRMS-ESI: |
| | m/z 370.0068, found [M + H]⁺ 371.0142; ¹H NMR (400 MHz, DMSO-d₆) δ |
| | 10.96 (s, 1H), 7.49 (s, 1H), 7.06 (s, 1H), 5.19 (dd, J = 12.8, 5.3 Hz, 1H), |
| | 3.97 (s, 3H), 3.96 (s, 3H), 2.92-2.82 (m, 1H), 2.62-2.57 (m. 1H), 2.48- |
| | 2.38 (m, 1H), 2.13-2.01 (m, 1H). |
| 12 | Compound 14, molecular formula: C₁₅H₁₆N₂O₆Se, yiled 65%. HRMS-ESI: |
| | m/z 400.0174, found [M + H]⁺ 401.0246; ¹H NMR (400 MHz, DMSO-d₆) δ |
| | 10.97 (s, 1H), 7.12 (s, 1H), 5.20 (dd, J = 12.8, 5.2 Hz, 1H), 3.97 (s, 3H), |
| | 3.96 (s, 3H), 3.95 (s, 3H), 2.91-2.83 (m, 1H), 2.56 (d, J = 17.3 Hz, 1H), |
| | 2.47-2.33 (m, 1H), 2.03-1.92 (m, 1H). |
| 13 | Compound 15, molecular formula: C₁₃H₁₂N₂O₃Se, yiled 87%. HRMS-ESI: |
| | m/z 324.0013, found [M + H]⁺ 325.0085; ¹H NMR (400 MHz, DMSO-d₆) δ |
| | 10.89 (s, 1H), 7.95 (d, J = 7.8 Hz, 1H), 7.41-7.27 (m, 2H), 5.24-4.73 (m, |
| | 1H), 2.88-2.43 (m, 3H), 2.34 (s, 3H), 2.08-1.92 (m, 1H). |
| 14 | Compound 16, molecular formula: C₁₃H₁₁N₃O₄Se, yiled 55%. MS-ESI |
| | [M + H]⁺ 354.0. |
| 15 | Compound 17, molecular formula: C₁₅H₁₄N₂O₄SSe. HRMS-ESI: m/z |
| | 397.9839, found [M + H]⁺ 398.9912; ¹H NMR (400 MHz, DMSO-d₆) δ 8.11 |
| | (d, J = 7.8 Hz, 1H), 7.88-7.63 (m, 2H), 7.47 (t, J = 7.4 Hz, 1H), 5.98 (s, |
| | 1H), 5.71 (s, 1H), 4.47 (s, 1H), 1.71 (s, 3H), 1.53 (s, 3H). |
| 16 | Compound 18, molecular formula: C₁₆H₁₄FNO₄SSe. HRMS-ESI: m/z |
| | 415.9745, found [M + H]⁺ 416.9818. |
| 17 | Compound 24, molecular formula: C₁₁H₈N₂O₃Se. HRMS-ESI: m/z |
| | 295.9700, found [M + H]⁺ 296.9773; ¹H NMR (400 MHz, DMSO-d₆) δ 11.41 |
| | (s, 1H), 8.06 (d, J = 8.1 Hz, 1H), 7.84-7.62 (m, 2H), 7.43 (t, J = 7.4 Hz, |
| | 1H), 5.30 (dd, J = 9.8, 5.7 Hz, 1H), 2.98 (dd, J = 18.0, 9.8 Hz, 1H), 2.86 |
| | (dd, J = 18.0, 5.7 Hz, 1H). |
| 18 | Compound 25, molecular formula: C₁₂H₁₀N₂O₄Se. HRMS-ESI: m/z |
| | 325.9806, found [M + H]⁺ 326.9879; ¹H NMR (400 MHz, DMSO-d₆) δ 11.36 |
| | (s, 1H), 7.90 (d, J = 8.6 Hz, 3H), 7.36-7.23 (m, 1H), 5.27 (dd, J = 9.8, 5.7 |
| | Hz, 1H), 3.87 (s, 3H), 2.95-2.82 (m, 2H). |
| 19 | Compound 26, molecular formula: C₁₁H₇FN₂O₃Se. HRMS-ESI: m/z |
| | 313.9606, found [M + H]⁺ 314.9682; ¹H NMR (400 MHz, DMSO-d₆) δ 11.41 |
| | (s, 1H), 8.06 (d, J = 8.1 Hz, 1H), 7.84-7.62 (m, 2H), 7.43 (t, J = 7.4 Hz, |
| | 1H), 5.30 (dd, J = 9.8, 5.7 Hz, 1H), 2.98 (dd, J = 18.0, 9.8 Hz, 1H), 2.86 |
| | (dd, J = 18.0, 5.7 Hz, 1H). |
| 20 | Compound 27, molecular formula: C₁₁H₇FN₂O₃Se. HRMS-ESI: m/z |
| | 313.9606, found [M + H]⁺ 314.9682; ¹H NMR (400 MHz, DMSO-d₆) ¹H NMR |
| | (400 MHz, DMSO-d₆) δ 11.42 (s, 1H), 8.07 (dd, J = 8.6, 5.2 Hz, 1H), 7.42- |
| | 7.31 (m, 2H), 5.33-5.28 (m, 1H), 2.98-2.83 (m, 2H). |
| 21 | Compound 28, molecular formula: C₁₂H₁₀N₂O₃Se. HRMS-ESI: m/z |
| | 309.9857, found [M + H]⁺ 310.9930; ¹H NMR (400 MHz, DMSO-d₆) δ 10.29 |
| | (s, 1H), 8.01 (d, J = 7.8 Hz, 1H), 7.37-7.23 (m, 1H), 5.33-5.25 (m, 1H), |
| | 2.96-2.81 (m, 2H), 2.35 (s, 3H). |

### Example 22. Synthesis of Compound 19

Synthesis Route:

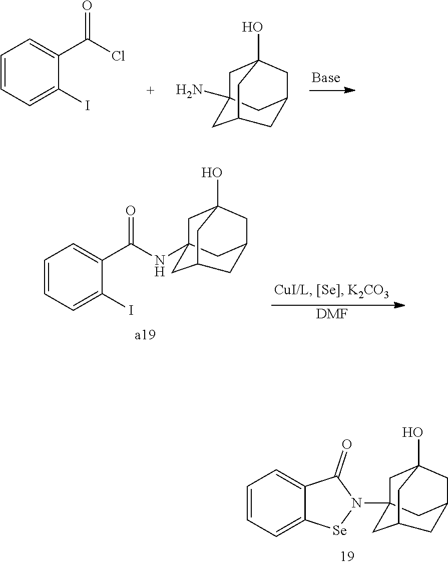

Step 1: Synthesis of Intermediate a19

To a stirred solution of 3-amino-1-adamantanol (167 mg, 1 mmol) and triethylamine (151 mg, 1.5 mmol) in tetrahydrofuran (10 mL) was slowly added o-iodobenzoyl chloride (266 mg, 1 mmol) under ice bath, and the resulting mixture was reacted for 1 to 2 h. After the reaction was completed (monitored by TLC), 20 ml of water was added, followed by extraction with ethyl acetate (20 mL×2). The combined organic phases were sequentially washed with saturated brine, dried over anhydrous sodium sulfate, and filtered. The resulting filtrate was evaporated under reduced pressure and purified by silica gel column chromatography (V_{ethyl acetate}:V_{petroleum ether}=1:4 to 1:1) to give the key intermediate a19. MS-ESI [M+H]⁺ 398.0.

Step 2: Synthesis of Compound 19

To a stirred solution of a19 (397 mg, 1 mmol), selenium powder (0.15 g, 1.9 mmol), and K₂CO₃(276 mg, 2 mmol) in DMF (5 mL) were added CuI (154 mg, 0.8 mmol), 1,10-o-diphenyl azaphenanthrene (146 mg, 0.8 mmol), and the resulting mixture was reacted at 110° C. for 24 hours under nitrogen atmosphere. After the reaction was completed (monitored by TLC), 20 ml of water was added, followed by extraction with ethyl acetate (20 mL×2). The combined organic phases were sequentially washed with saturated brine, dried over anhydrous sodium sulfate, and filtered. The resulting filtrate was evaporated under reduced pressure and purified by silica gel column chromatography (V_{acetone}:V_{petroleum ether}=1:4 to 1:1) to give compound 19 (251 mg, yield 72%). HRMS-ESI: m/z C₁₇H₁₉NO₂Se: 349.0581, found [M+H]⁺ 350.0655; ¹H NMR (400 MHz, CDCl₃) δ 8.04 (d, J=8.2 Hz, 1H), 7.83 (d, J=7.4 Hz, 1H), 7.68-7.60 (m, 1H), 7.43 (t, J=7.4 Hz, 1H), 2.48-1.60 (m, 15H).

Example 23 to 29 were performed according to the operation of example 22, wherein examples 24 to 26 were synthesized using 2-amino-benzothiophene instead of 3-amino-1-amantadine, examples of results obtained are as follows:

| Examples | Compound information |
|---|---|
| 23 | Compound 20, molecular formula: C₁₇H₁₈FNO₂Se, [M + H]⁺ 368.1. |
| 24 | Compound 21, molecular formula: C₁₄H₈N₂OSSe, HRMS-ESI: m/z |
| | 331.9523, found [M + H]⁺ 332.9595; ¹H NMR (400 MHz, DMSO-d₆): δ 8.16 |
| | (dd, J = 7.4, 3.1 Hz, 1H), 8.07-7.92 (m, 5H), 7.57 (t, J = 7.4 Hz, 1H), 7.48 |
| | (t, J = 7.4 Hz, 1H). |
| 25 | Compound 22, molecular formula: C₁₅H₇F₃N₂O₂SSe, HRMS-ESI: m/z |
| | 415.9346, found [M + H]⁺ 416.9418; ¹H NMR (400 MHz, DMSO-d₆) δ 8.20 |
| | (s, 1H), 8.15 (d, J = 8.1 Hz, 1H), 8.04 (d, J = 7.4 Hz, 1H), 7.96 (d, J = 8.8 |
| | Hz, 1H), 7.79 (t, J = 7.5 Hz, 1H), 7.55 (t, J = 7.4 Hz, 1H), 7.48-7.38 (m, 1H). |
| 26 | Compound 23, molecular formula: C₁₅H₁₀N₂O₂SSe, MS-ESI [M + H]⁺ 363.0. |
| 27 | Compound 29, molecular formula: C₁₅H₁₀N₂OSSe, MS-ESI [M + H]⁺ 346.9. |
| 28 | Compound 30, molecular formula: C₁₆H₉F₃N₂O₃SSe, MS-ESI [M + H]⁺ |
| | 446.9. |
| 29 | Compound 31, molecular formula: C₁₅H₁₀FN₂O₂SSe, MS-ESI [M + H]⁺ |
| | 380.9. |

### Example 30. Synthesis of Compound 3

To a solution of compound 2 (72 mg, 0.2 mmol) in methanol (1 mL) was added 10% Pd/C, and the resulting mixture was heated to 70° C. under H₂ atmosphere for 48 hours. After the reaction was completed (monitored by TLC), the mixture was filtered, the obtained filtrate was evaporated under reduced pressure and purified by column chromatography to give compound 6 (25 mg, 36%). HRMS-ESI: m/z calcd for C₁₂H₁₀N₂O₃Se: 324.9966, found [M+H]⁺ 326.0040; ¹H NMR (400 MHz, DMSO-d₆) δ 10.97 (s, 1H), 7.47-7.34 (m, 1H), 7.05-6.83 (m, 2H), 6.33 (brs, 2H), 5.23 (dd, J=12.8, 5.3 Hz, 1H), 2.92-2.83 (m, 1H), 2.59 (d, J=17.3 Hz, 1H), 2.45-2.35 (m, 1H), 2.12-2.02 (m, 1H).

### Example 31. Synthesis of Compound 6

To a solution of compound 5 (72 mg, 0.2 mmol) in methanol (1 mL) was added 10% Pd/C and 80% hydrazine hydrate (40 mg) under ice bath and nitrogen atmosphere, and the reaction was heated to 40° C. for 8 hours. After the reaction was completed (monitored by TLC), the mixture was filtered, the obtained filtrate was evaporated under reduced pressure and purified by column chromatography to give compound 6 (15 mg, 22%). HRMS-ESI: m/z calcd for C₁₂H₁₁N₃O₃Se: 324.9966, found [M+H]⁺ 326.0040; ¹H NMR (400 MHz, DMSO-d₆) δ 10.97 (s, 1H), 7.29-6.91 (m, 3H), 6.12 (brs, 2H), 5.21 (dd, J=12.8, 5.3 Hz, 1H), 2.94-2.82 (m, 1H), 2.59-2.52 (m, 1H), 2.43-2.29 (m, 1H), 2.12-2.01 (in, 1H).

### Example 32. Synthesis of Compound 32

To a stirred solution of compound 19 (35 mg, 0.1 mmol) in methanol (2 ml) was slowly added 30% hydrogen peroxide (0.12 mmol) under ice bath, the reaction temperature was raised to room temperature and performed for 12 hours. After the reaction was completed (monitored by TLC), 5 ml of water was added, and then ethyl acetate (5 mL×2) was added for extraction. The organic phase was sequentially washed with saturated brine, dried over anhydrous sodium sulfate, and filtered. The resulting filtrate was evaporated under reduced pressure and purified by silica gel column chromatography (V_{ethyl acetate}:V_{petroleum ether}=1:1) to produce compound 32 (27 mg, yield 75%). HRMS-ESI: m/z calcd for C₁₇H₁₉NO₃Se: 365.0530, found [M+H]⁺ 366.0609; ¹H NMR (400 MHz, DMSO-d₆) δ8.24-7.96 (m, 2H), 7.83-7.62 (m, 2H), 2.76-1.71 (n, 15H).

Example 33 to 42 were performed according to the operation of example 32, examples of results obtained are as follows:

| Examples | Compound information |
|---|---|
| 33 | Compound 33, yiled 26%, molecular formula: C₁₄H₈N₂O₂SSe, HRMS-ESI: m/z |
| | 347.9472, found [M + H]⁺ 348.9548; ¹H NMR (400 MHz, DMSO-d₆): δ |
| | 8.18-7.95 (m, 6H), 7.62 (t, J = 7.1 Hz, 1H), 7.52 (t, J = 7.1 Hz, 1H). |
| 34 | Compound 34, yiled 21%, molecular formula: C₁₅H₇F₃N₂O₃SSe, MS-ESI |
| | [M + H]⁺ 432.9. |
| 35 | Compound 36, yiled 43%, molecular formula: C₁₂H₉FN₂O₄Se, MS-ESI |
| | [M + H]⁺ 345.0. |
| 36 | Compound 37, yiled 47%, molecular formula: C₁₄H₁₃N₃O₅Se, MS-ESI |
| | [M + H]⁺ 384.0. |
| 37 | Compound 38, yiled 35%, molecular formula: C₁₃H₉N₃O₄Se, MS-ESI |
| | [M + H]⁺ 352.0. |
| 38 | Compound 39, yiled 29%, molecular formula: C₁₂H₉FN₂O₄Se, HRMS-ESI: |
| | m/z 343.9712, found [M + H]⁺ 344.9784; ¹H NMR (400 MHz, DMSO-d₆): δ |
| | 11.08 (s, 1H), 8.12-8.08 (m, 1H), 7.42-7.23 (m, 2H), 5.31-5.27 (m, 1H), 2.96- |
| | 2.87 (m, 1H), 2.64-2.53 (m, 1H), 2.48-2.35 (m, 1H), 2.17-2.02 (m, 1H). |
| 39 | Compound 40, yiled 51%, molecular formula: C₁₃H₁₂N₂O₅Se, MS-ESI |
| | [M + H]⁺ 357.0. |
| 40 | Compound 41, yiled 39%, molecular formula: C₁₁H₈N₂O₄Se, HRMS-ESI: |
| | m/z 311.9649, found [M + H]⁺ 312.9724; ¹H NMR (400 MHz, DMSO-d₆): δ |
| | 11.49 (s, 1H), 8.28-7.82 (m, 3H), 7.59 (t, J = 7.4 Hz, 1H), 5.33-5.27 (m, 1H), |
| | 3.02-2.95 (m, 1H), 2.89-2.82(m, 1H). |
| 41 | Compound 42, yiled 42%, molecular formula: C₁₂H₁₀N₂O₄Se, MS-ESI |
| | [M + H]⁺ 327.0. |
| 42 | Compound 43, yiled 34%, molecular formula: C₁₂H₁₀N₂O₅Se, MS-ESI |
| | [M + H]⁺ 343.0. |

### Example 43. Synthesis of Compound 35

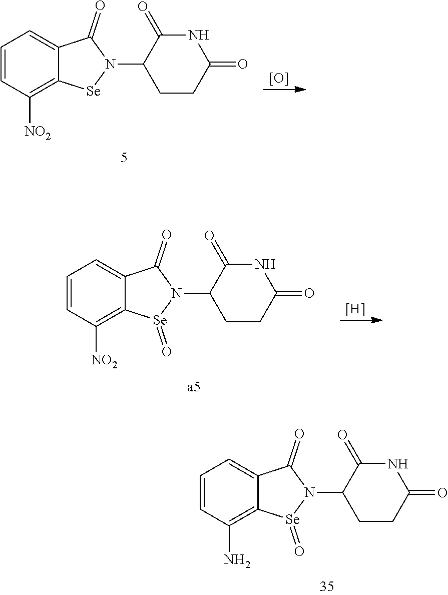

Compound a5 could be obtained through a peroxidation according to the synthesis method described in example 32. To a stirred solution of compound a5 (37 mg, 0.1 mmol) in THF (1 mL) was slowly added an aqueous solution (0.7 ml) of sodium bisulfite (52 mg, 0.5 mmol) under nitrogen atmosphere, and the reaction was performed at 60° C. After the reaction was completed (monitored by TLC), the reaction mixture was cooled to room temperature, and extracted with ethyl acetate (5 mL×2). The combined organic phases were sequentially washed with saturated brine, dried over anhydrous sodium sulfate, and filtered. The obtained filtrate was evaporated under reduced pressure to give compound 35 (27 mg, yield 79%). Molecular formula: C₁₂H₁₁N₃O₄Se, HRMS-ESI: m/z calcd for 340.9915, found [M+H]⁺ 341.9984.

### Example 44. Synthesis of Compound 44

### Step 1: Preparation of 2,2′-Diselenylbisbenzaldehyde

3-Methoxy-2-aminobenzaldehyde (1.51 g, 10 mmol) was added to a 3N hydrochloric acid water-DMSO (V:V=1:1) mixed solution (4 mL) under an ice bath, and then sodium nitrite (10 mmol, 690 mg) in an aqueous solution (2 ml) was slowly added to the reaction mixture under stirring, and the reaction will change to a clear solution after an hour and to give the desired diazonium salt solution.

Selenium powder (790 mg, 10 mmol) was mixed with cetyltrimethyl ammonium bromide (20 mg) and 2N aqueous sodium hydroxide solution (5 ml) to obtain a Se—NaOH solution under nitrogen atmosphere. NaBH₄ (49 mg, 1.3 mmol) solution (1 mL) that contained NaOH (40 mg, 1 mmol) was added drop-wisely into the Se—NaOH solution under an ice bath. The reaction was stirred at room temperature for 1 h and then at 90° C. for 0.5 h to from Na₂Se₂. After cooling to the room temperature, the diazonium salt solution obtained above was slowly added dropwise to the Na₂Se₂ solution, and the mixture was heated to 40° C. for 2 hours. After the reaction was completed, the reaction mixture was acidified to neutral by adding 1N HCl, and then extracted with ethyl acetate for two times, the combined organic phases were evaporated under reduced pressure and purified by silica gel column chromatography (eluted with dichloromethane and methanol) to produce a khaki solid of 2,2′-diselenylbisbenzaldehyde in 45% yield. Molecular formula: C₁₆H₁₄O₄Se₂, HRMS-ESI: m/z calcd for 428.9223, found [M+H]⁺ 430.9298; ¹H NMR (CDCl₃), δ (ppm): 3.78 (s, 6H), 7.09-7.13 (dd, J=7.6 Hz, 2H), 7.42-7.50 (m, 4H), 10.20 (s, 2H).

### Step 2: Synthesis of Target Compound 44

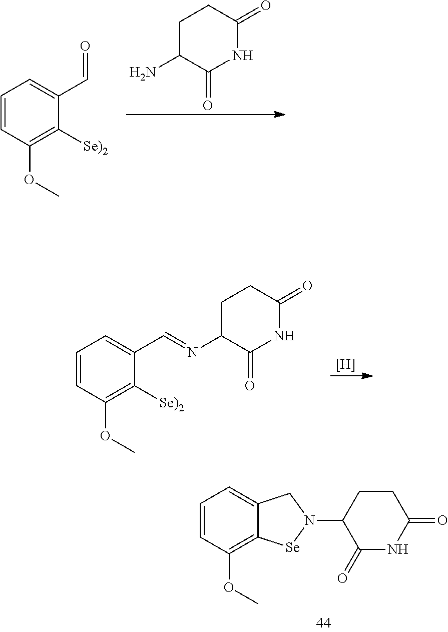

To a stirred solution of 2,2′-diselenylbisbenzaldehyde (429 mg, 1 mmol) in 20 ml of acetonitrile was added 50 μL of conc HCl and stirred for 15 min, then 3-amino-2,6-piperidinedione (128 mg, 1 mmol) was added and stirred for 6 hours to obtain the enamine intermediate. The above reaction mixture was concentrated under reduced pressure and redissolved with 10 ml of methanol, followed by adding sodium borohydride (38 mg, 1 mmol) under ice bath and the reaction was performed for another 6 h. After the reaction is completed, the resulting mixture was extracted with ethyl acetate, dried over magnesium sulfate, filtered, concentrated and purified by silica gel column chromatography to give compound 44 in 36% yield, 117 mg. HRMS-ESI: m/z calcd for C₁₃H₁₄N₂O₃Se: 326.0170, found [M+H]⁺ 327.0245; ¹H NMR (DMSO-d₆), δ (ppm): 10.91 (s, 1H), 7.85-7.69 (t, J=7.8 Hz, 1H), 7.58-7.31 (m, 2H), 4.75-4.62 (m, 3H), 3.89 (s, 3H), 2.78-2.42 (m, 2H), 2.31-1.91 (m, 2H).

Examples 45 to 48 were performed according to the operation of example 44, examples of results obtained are as follows:

| Examples | Compound information |
|---|---|
| 45 | Compound 48, yiled 23%, molecular formula: C₁₆H₁₁F_{3N2}O₂SSe, |
| | HRMS-ESI: m/z 431.9659, found [M + H]⁺ 432.9730; ¹H NMR (400 MHz, |
| | DMSO-d₆): δ 8.06-7.35 (m, 6H), 4.86 (s, 2 H), 3.92 (s, 3 H). |
| 46 | Compound 49, yiled 41%, molecular formula: C₁₆H₁₈F₃N₂O₄SSe, |
| | MS-ESI [M + H]⁺ 415.0. |
| 47 | Compound 50, yiled 49%, molecular formula: C₁₂H₉FN₂O₄Se, |
| | HRMS-ESI: m/z 365.0894, found [M + H]⁺ 366.0967; ¹H NMR (400 MHz, |
| | CDCl₃): δ 7.21-7.15 (t, J = 7.8 Hz, 1H), 6.93-6.73 (m, 1H), 4.40 (s, 2H), |
| | 3.84 (s, 1H), 2.43-1.51 (m, 15H). |
| 48 | Compound 51, yiled 34%, molecular formula: C₁₃H₁₂N₂O₅Se, |
| | HRMS-ESI: m/z 312.0013, found [M + H]⁺ 313.0086. |

### Effect Examples

### Example 49. TNF-α Activity Inhibiting Assay

Methods: Peripheral blood from healthy volunteers was collected with EDTA anticoagulant tubes. After being diluted 5-fold with 1640 medium (Gibco, USA), the blood was added to 96-well cell culture plates (Costar, USA) and then treated with 10 μL solution of the compound of general formula (I) of the present invention in DMSO (Sigma, USA), and the final concentration of DMSO was 0.2%. After incubation for 60 minutes in an incubator at 37° C. under 5% CO₂, 10 μL LPS (Sigma, USA) was added to the reaction system, and the final concentration was 10 ng/mL. After further culturing for 6 hours in the incubator at 37° C. under 5% CO₂, the supernatant was collected. The content of TNF-α was determined by ELISA (BD Biosciences, USA). Absorbance was detected at OD₄₅₀ nm with a microplate reader, with GD 650 nm as reference. The control, a solution containing 0.2% DMSO medium, was as 0% inhibition. Raw data and standard curves were recorded. The four-parameter drug inhibition curve was plotted by XL-fit software and the inhibition rate of each compound was calculated. The experimental results are shown in table 1.

TABLE 1
TNF-α inhibitory activity

| Comps | TNF-α inhibition (%) |
|---|---|
| 1 | C |
| 2 | B |
| 3 | A |
| 4 | C |
| 5 | B |
| 6 | A |
| 7 | C |
| 8 | C |
| 9 | C |
| 10 | B |
| 11 | B |
| 12 | B |
| 13 | B |
| 14 | A |
| 15 | A |
| 16 | B |
| 17 | D |
| 18 | D |
| 19 | C |
| 20 | C |
| 21 | C |
| 22 | B |
| 23 | C |
| 24 | C |
| 25 | C |
| 26 | C |
| 27 | B |
| 28 | A |
| 29 | B |
| 30 | C |
| 31 | D |
| 32 | D |
| 33 | D |
| 34 | C |
| 35 | A |
| 36 | C |
| 37 | C |
| 38 | A |
| 39 | C |
| 40 | B |
| 41 | C |
| 44 | B |
| 50 | C |
| Thalidomide | D |
| Lenadomide | A |

Note:
A: <1 μM; B: 1~10 μM; C: 10~100 μM; D: >100 μM.

### Example 50 Thioredoxin Reductase 1 (TrxR1) Activity Inhibiting Assay

Working solutions: 0.119 mg/mL of TrxR working solution preparation: 175 μL of TrxR stock solution (0.34 mg/mL) was diluted to 500 μL; 1 mM NADPH working solution preparation, NADPH (5 mg) was dissolved in 12 mL potassium phosphate buffer; 1 mM DTNB working solution preparation: 25 mg DTNB was dissolve in 63 mL DMSO; potassium phosphate buffer system preparation: 0.2 mg/mL bovine serum albumin (BSA) and 1 mM EDTA were added to potassium phosphate buffer at pH 7.4 (K₂HPO₄/KH₂PO₄).

Methods: The TrxR1 inhibitory activity study of selenium-containing isoxazolamines in vitro. Insulin, NADPH, Trx, the samples and TrxR1 were added to a microcuvette in order, and subsequently added the work buffer (0.1 mol/L potassium phosphate/2 mmol/L EDTA) to a total volume of 0.5 mL. The concentration of each component in the obtained reaction system is: insulin 130 μmol/L, NADPH 0.4 mol/L (Sigma), Trx 4 μmol/L, the added substrate (the selenium-containing isoxazolamines treated samples). After determination of the initial absorption at 340 nm, the enzymatic reaction was started by adding 20 μg extracted TrxR1 proteins, the decrease in absorbance at 340 nm was monitored. One unit of enzymatic activity is defined as the consumption. The activity can be calculated from the extinction coefficient of NADPH, and the unit of enzyme activity is defined as: 1U=ΔA340 nm/min×1000, the TR activity could be calculated with U/L. The results are shown in table 2.

TABLE 2
TrxR inhibition activity of compounds of formula I

| Comps | TrxR inhibition IC₅₀ |
|---|---|
| 1 | B |
| 2 | B |
| 4 | A |
| 5 | C |
| 7 | A |
| 8 | B |
| 9 | B |
| 10 | A |
| 12 | A |
| 13 | A |
| 16 | B |
| 17 | A |
| 21 | B |
| 22 | A |
| 23 | B |
| 25 | B |
| 27 | B |
| 30 | A |
| 31 | B |
| 34 | C |
| 38 | D |
| 40 | C |
| 45 | B |
| Ebselen | C |

Note:
A: <5 μM; B: 5~50 μM; C: 50~100 μM; D: >100 μM.

### Example 51: Glutathione Peroxidase (GPx)-Like Activity Assay

Method: GPX activity of selenium compounds was assayed by ultraviolet spectrophotometry. Glutathione (2.0 mM), EDTA (1 mM), glutathione disulfide reductase (1.7 units mL⁻¹), and nicotinamide adenine dinucleotide phosphate oxidase (NADPH; 0.4 mM) were mixed intopotassium phosphate buffer (0.1 M, pH=7.5). The selenium compounds (50 μm) was added to the above mixture At room temperature (25° C.), and the reaction was started by the addition of H₂O₂, tBuOOH or Cum-OOH (1.6 mM) respectively. The initial reduction rates were calculated from the rate of NADPH oxidation at 340 nm in a GSH assay. Each initial rate was measured at least three times and calculated from the first 5-10% of the reaction by using 6.22 mM⁻¹ cm⁻¹ as the molar extinction coefficient for NADPH. For the peroxidase activity, the rates were corrected for the background reaction between peroxide and thiol. The results are shown in table 3.

TABLE 3
Anti-peroxidation effects of selenium-containing
isoxazolamines represented as formula I

| | Initial rate v₀ [μm min⁻¹] | | |
|---|---|---|---|
| Comps | H₂O₂ | tBuOOH | Cum-OOH |
| 1 | 109.7 ± 2.1 | 42.0 ± 3.3 | 124.5 ± 7.6 |
| 7 | 128.2 ± 2.0 | 49.3 ± 5.0 | 142.2 ± 8.2 |
| 12 | 121.7 ± 2.2 | 54.5 ± 5.7 | 116.8 ± 7.2 |
| 19 | 99.7 ± 1.9 | 32.3 ± 3.5 | 34.2 ± 1.5 |
| 21 | 115.3 ± 2.6 | 35.7 ± 4.2 | 94.5 ± 4.2 |
| 26 | 145.2 ± 3.4 | 55.7 ± 5.2 | 164.5 ± 9.1 |
| 32 | 36.7 ± 0.8 | 12.0 ± 2.3 | 11.5 ± 0.2 |
| 34 | 39.7 ± 1.4 | 14.2 ± 1.1 | 12.4 ± 0.3 |
| Ebselen | 76.7 ± 1.1 | 21.0 ± 4.3 | 31.2 ± 0.5 |

The results showed that some of the selenium-containing isoxazolamines had better anti-peroxidation activities than the positive control ebselen.

### Example 52 Effect on Endothelial Cell Damage Caused by Ox-LDL

Drugs: Selenium-containing isoxazolamines were dissolved in DMSO.

Reagents: Ox-LDL (Beijing Xiehe Sanyou); DMEM medium (Low sugar, GIBCO, UK); HMEC cells (Shanghai Institute of Biochemistry and Cell Biology, Chinese Academy of Sciences); MTT (Sigma, product No. 5655, USA); other reagents are analytical grade.

Method: HMEC cells were cultured in DMEM media supplemented with 10% FBS at 37° C. with 5% CO₂. The MTT assay was used for the determination of cytotoxicity. The cells were harvested at logarithmic phase and plated at a density of 2×10⁴ cells per well in 96-well plates, cultured for 48 hours and grew into a tightly packed single molecular layer. Using the serum-free DMEM medium instead, and 100 μL aliquots of medium containing 5 μM of the selenium-containing isoxazolamines or contrast were added subsequently. After incubating for 1 hour, 100 μg/ml ox-LDL was added to the injured groups. After 24 h of incubation, cell viability was measured using the 3-(4,5-dimethylthiazol-2-yl)-2,5-diphenyltetrazolium bromide (MTT) assay. In brief, MTT (100 μL, 0.5 mg mL⁻¹) was added to the wells for 4 h at 37° C., discarded the culture medium, DMSO 150 μL/well was added and shaked for 5 minutes to release the dye, and measured the OD at 570 nm with a microplate reader. The changes in OD caused by cytokine and drug treatment were used as an index of cell viability, normalised to cells incubated in control medium, which were considered 100% viable.

Results: As shown in FIG. 2, ox-LDL could cause HMEC cells damage, and the selenium-containing isoxazolamines could significantly inhibit HMEC cells death caused by ox-LDL.

### Example 53. Effect of the Selenium-Containing Isoxazolamines on Erastin-Induced Ferroptosis in HT22 Cells

Drugs: Selenium-containing isoxazolamines and erastin, which could be dissolved by DMSO.

Reagents: CCK-8 kit (Sigma, USA), DEME medium (Sigma, USA), mouse HT22 hippocampal cells (Shanghai Jiaotong University).

Method: HT22 cells were cultured in DMEM media supplemented with 10% fetal bovin serum at 37° C. with 5% CO₂. The culture HT22 cells were plated in 96-well plates and allowed to incubate for 24 hours. Subsequently, 100 μL aliquots of medium containing 5 μM of the selenium-containing isoxazolamines was added and cultured for 2 hours. Afterward, 0.5 μmol/L Erastin was added and cultured for 8 hours, and then added 10 μL of the CCK-8 solution per well and incubated for 3 hours, and measured the OD at 450 nm with a microplate reader. The cell survival rate was calculated according to the following formula: cell survival rate %=(treatment group-blank control group)/(control group-blank control group)*100%. The experiment was repeated three times.

Results: As shown in FIG. 3, erastin could cause apoptosis in HT22 cells, and the selenium-containing isoxazolamines could significantly reduce the erastin-induced ferroptosis and improve cell survival rate.

1. A selenium-containing isoxazolamine derivative having a structure of general formula (I), a pharmaceutically acceptable salt, a solvate, a polymorph, a stereoisomer, an isotopic compound, or a metabolite thereof; 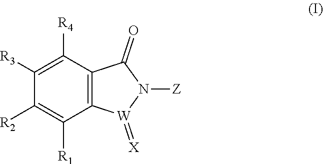
in the general formula (I),
each of R₁, R₂, R₃ and R₄ is independently selected from H, D, halogen, hydroxyl, amino, nitro, cyano, carboxyl, seleno, mercapto, (C₁-C₈) alkylselenyl, (C₁-C₈) alkylselenyl (C₁-C₈) alkylamino, (C₂-C₈) alkenylselenyl, α-(C₁-C₈) alkylselenyl amino acid, α-(C₁-C₈) alkylselenyl formyl amino acid, (C₀-C₈) alkylamino (C₁-C₈) alkylselenyl, (C₀-C₈) alkylaminoformylselenyl, (C₀-C₈) alkylaminoformyl, arylselenyl, (C₀-C₈) alkoxyl (C₁-C₈) alkylselenyl, (C₀-C₈) alkoxyformyl (C₁-C₈) alkylselenyl, (C₀-C₈) alkoxyformyl C₁-C₈ alkoxyl, halo (C₁-C₈) alkylselenyl, C₁-C₈ alkanesulfonyl, (C₁-C₈) alkanesulfonamido, (C₀-C₈) alkylaminosulfonyl, (C₁-C₈) alkyl, halo (C₁-C₈) alkyl, halo (C₁-C₈) alkoxyl, (C₀-C₈) alkylethynyl, (C₁-C₈) alkoxyl, (C₁-C₈) alkylacyloxy, (C₁-C₈) alkoxyl (C₁-C₈) alkoxyl, (C₁-C₈) alkoxyl (C₁-C₈) alkyl, (C₁-C₈) alkylamino, (C₀-C₈) alkylamino (C₁-C₈) alkyl, aryl, aryl (C₁-C₈) alkylamino (C₁-C₈) alkyl, amidino, guanidino, arylsulfonamido, arylaminosulfonyl, benzoyl, (C₀-C₈) alkylselenyl formyl, aryl (C₁-C₈) alkylamino, aryl (C₁-C₈) alkylamido, (C₁-C₈) alkoxyformyl, (C₁-C₈) alkylamido, (C₁-C₈) alkylamino, (C₀-C₈) alkylselenyl formamido, arylselenyl (C₁-C₈)alkylamido, selenylcyano (C₁-C₈) alkylamido, benzoisoselenidazolone amino, benzoisoselenidazolone amino (C₁-C₈) alkylamido, benzoisoselenidazolone amino (C₁-C₈) alkanesulfonamido, (C₀-C₈) alkylamino selenyl, (C₀-C₈) alkylaminoformyl, (C₀-C₈) alkylamino formylselenyl, (C₁-C₈) alkylaminoformyloxyl, (C₁-C₈) alkylaminoformyl, (C₁-C₈) alkylaminoformyloxy, arylaminoformamido, arylaminoformyl, arylaminoformyloxy, piperidinyl, piperazinyl, morpholinyl, pyrrolyl, pyrazolyl, imidazolyl, pyridyl, pyrazinyl, quinolinyl, pyrimidinyl, pyrimidinylamino, thiazolyl, thienyl, furanyl, pyrrolyl or absent; wherein, the aryl groups of R₁, R₂, R₃ and R₄ described are phenyl or are phenyl which independently substituted with 1-4 halogen, hydroxy, nitro, cyano, amino, trifluoromethyl, carboxyl, (C₀-C₈) alkylaminosulfonyl, (C₁-C₈) alkanesulfonamido, (C₁-C₈) alkyl, halo (C₁-C₈) alkoxyl, (C₁-C₈) alkoxyl groups; wherein, the benzoisoselenidazolone amino described is 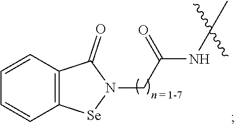
Z is: 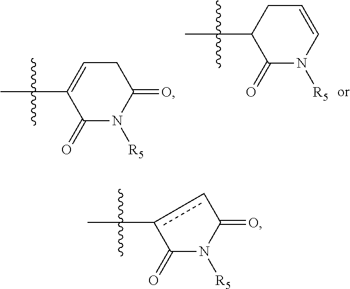
wherein Z is 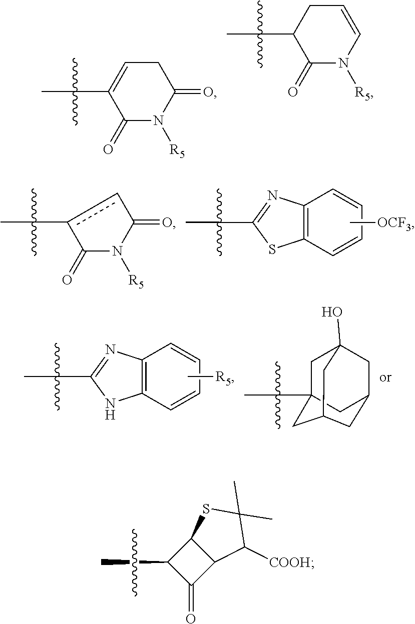
R₅ is selected from H, D, (C₁-C₈) alkylselenyl (C₁-C₈) alkyl, (C₂-C₈) alkenylselenyl (C₁-C₈) alkyl, selenocyanate (C₁-C₈) alkyl, 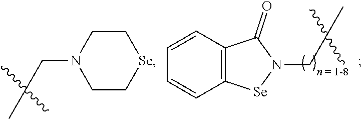
wherein Z is s 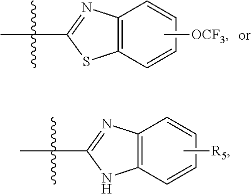
R₅ is selected from H, halogen, hydroxyl, nitro, cyano, amino, trifluoromethyl, carboxyl, (C₁-C₈) alkanesulfonyl, amino sulfonyl, (C₁-C₈) alkyl, halo (C₁-C₈) alkoxyl, (C₁-C₈) alkoxyl;
W is C or Se; wherein, when W is C, there is one selenium-containing substituent exists in the R₁, R₂, R₃, R₄, and R₅ group at least; and when W is Se, R₁, R₂, R₃, R₄, and R₅ could be any substitutes as described above;
X is, O or not exist;
Where bonds represented by [symbol] is chemical bond or not exist.

2. The selenium-containing isoxazolamine derivatives having a structure of general formula (I), the pharmaceutically acceptable salt, the solvate, the polymorph, the stereoisomer, the isotopic compound, or the metabolite thereof according to claim 1, wherein the compounds having structures of general formula (I-a), (I-b), (I-c), (I-d) and/or (I-e): 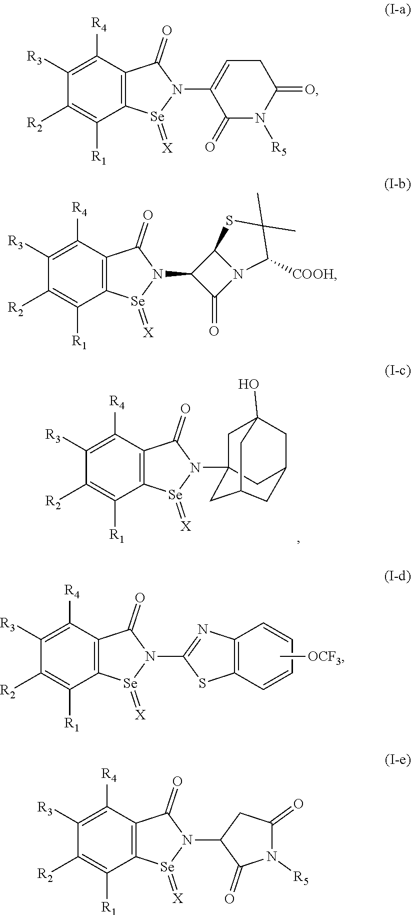
in the general formula (I-a˜I-e), each of R₁, R₂, R₃ and R₄ is independently selected from H, D, halogen, hydroxyl, amino, nitro, cyano, carboxyl, (C₀-C₈) alkylamino (C₁-C₈) alkylselenyl, (C₀-C₈) alkylaminoformyl (C₁-C₈) alkoxyl, amidino, guanidino, C₁-C₈ alkanesulfonyl, (C₁-C₈) alkanesulfonamido, (C₀-C₈) alkylaminosulfonyl, (C₁-C₈) alkyl, halo (C₁-C₈) alkyl, halo (C₁-C₈) alkoxyl, (C₀-C₈) alkylethynyl, (C₁-C₈) alkoxyl, (C₁-C₈) alkylacyloxy, (C₁-C₈) alkoxyl (C₁-C₈) alkoxyl, (C₁-C₈) alkoxyl (C₁-C₈) alkyl, (C₁-C₈) alkylamino, (C₀-C₈) alkylamino (C₁-C₈) alkyl, aryl, aryl (C₁-C₈) alkylamino (C₁-C₈) alkyl, arylsulfonamido, arylaminosulfonyl, benzoyl, arylmethylamino, aryl formamido, (C₀-C₈) alkoxyformyl, (C₁-C₈) alkylamido, (C₁-C₈) alkylamino, (C₀-C₈) alkylaminoformamido, (C₀-C₈) alkylaminoformyl, arylaminoformamido, arylaminoformyloxy or absent; wherein, the aryl groups of R₁, R₂, R₃ and R₄ described are phenyl or are phenyl which independently substituted with 1-4 halogen, hydroxy, nitro, cyano, trifluoromethyl, carboxyl, aminosulfonyl, (C₁-C₆) alkyl, (C₁-C₆) alkoxyl groups;
X is, O or not exist;
where bonds represented by [symbol] is chemical bond or not exist.

3. The selenium-containing isoxazolamine derivatives having a structure of general formula (I), the pharmaceutically acceptable salt, the solvate, the polymorph, the stereoisomer, the isotopic compound, or the metabolite thereof according to claim 1, wherein the compounds having a structure of general formula (I) is selected from the group consisting of

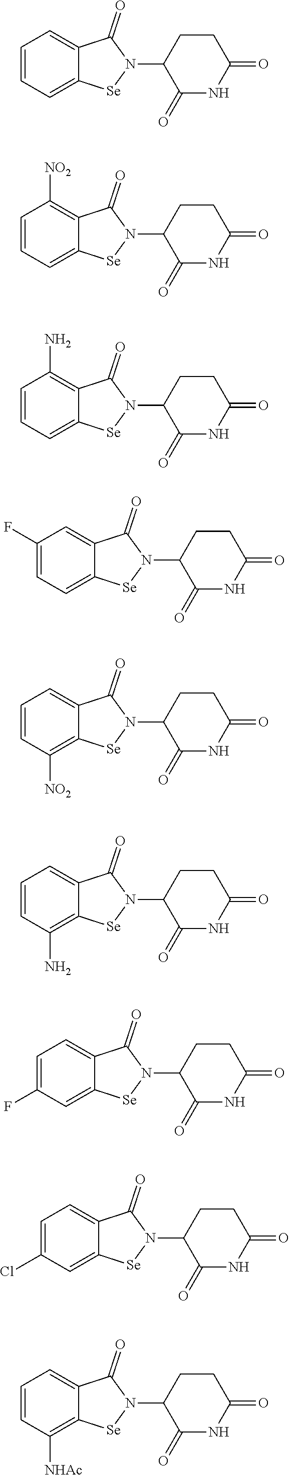

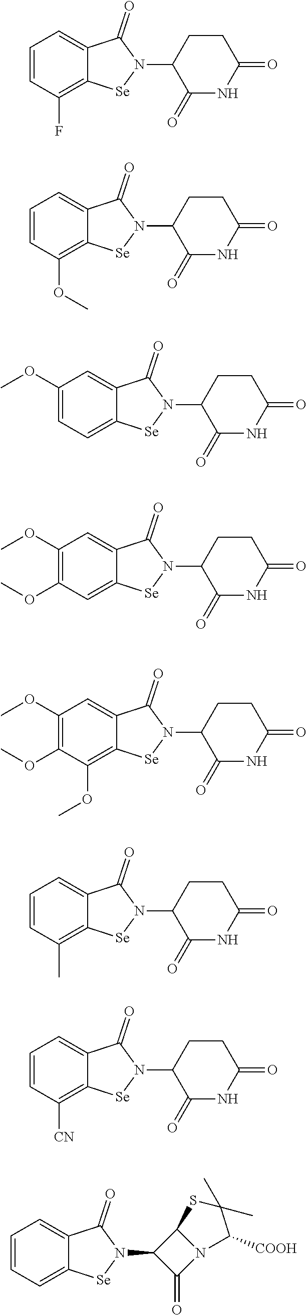

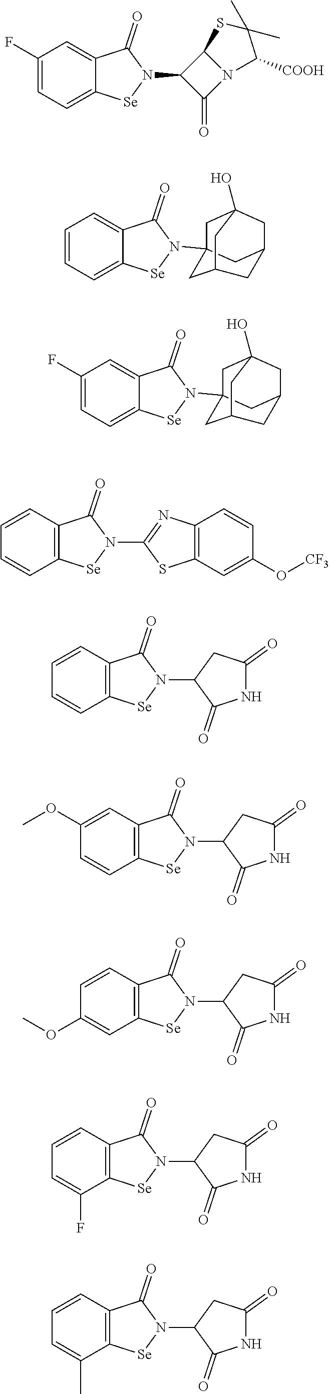

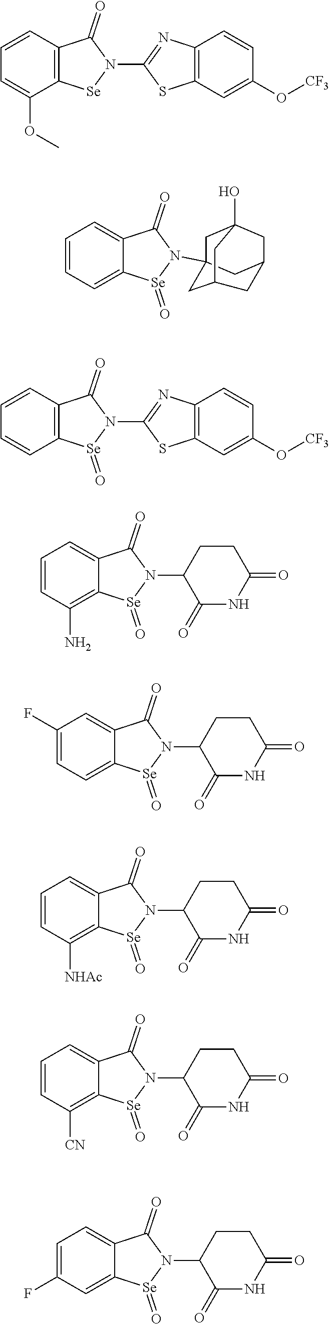

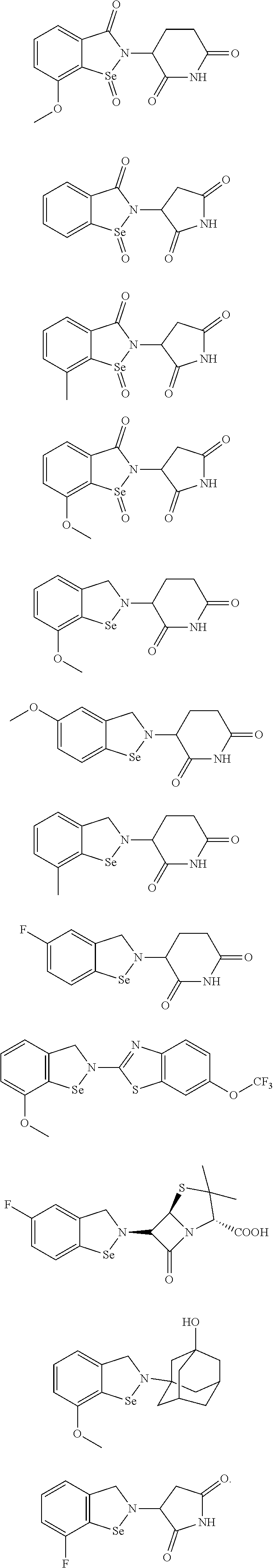

4. A pharmaceutical composition, which comprises at least one substance selected from the group consisting of the selenium-containing isoxazolamine derivative of claim 1, the pharmaceutically acceptable salt, the solvate, the polymorph, the stereoisomer, the isotopic compound, or the metabolite thereof, as well as one or more pharmaceutically acceptable carriers, diluents or excipients.

5. A process for preparing the selenium-containing isoxazolamine derivatives having a structure of general formula (I) according to claim 1, comprising:
(1) the first synthesis route for preparing some of the selenium-containing isoxazolamines represented by general formula I-a, I-b, I-c, I-d, and I-e 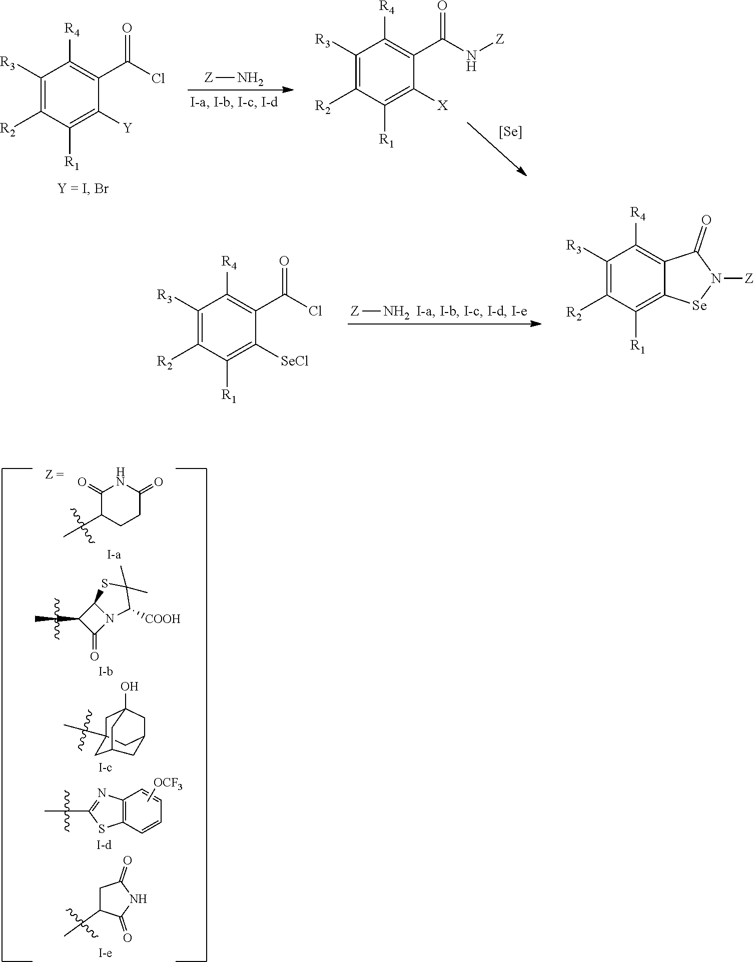
(2) the second synthesis route for preparing some of the selenium-containing isoxazolamines represented by general formula I-a, I-b, I-c, I-d, and I-e 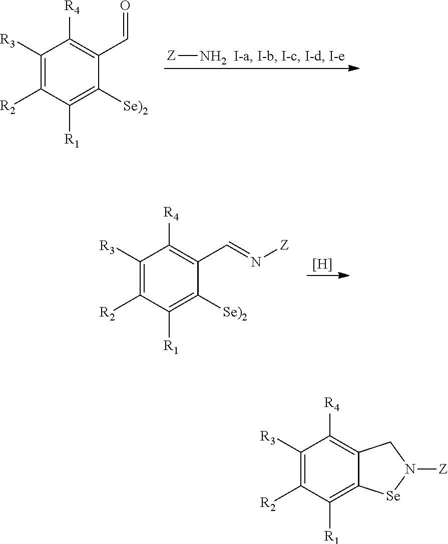
(3) the third synthesis route for preparing tetravalent selenium compounds represented by general formula I-a, I-b, I-c, I-d, and I-e 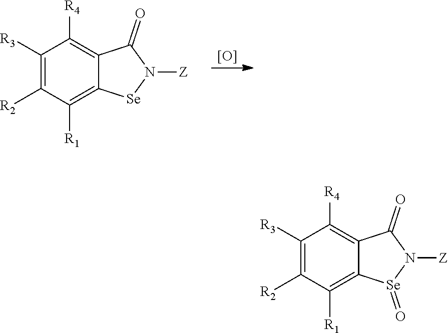
in the first synthesis route for preparing the selenium-containing isoxazolamines represented by general formula I-a, I-b, I-c, I-d, and I-e, the series of ortho-SeCl-substituted benzoyl chlorides can react with different 3-amino-2,6-piperidinedione or 3-amino-1,4-dihydropyridine-2-(1H)-one or 3-amino-1-adamantanol or 2-benzothiazolamine or 3-amino-2,5-pyrroledione respectively to afford the corresponding desired products of formulas I-a, I-b, I-c, I-d, while using the series of ortho-I or Br-substituted of benzoyl chlorides as the starting substrates will obtain the o-halobenzamide intermediates firstly, which can react with [Se] reagents furtherly to produce the desired benzoisoselazolidone derivatives of formulas I-a, I-b, I-c, I-d and I-e;
in the second synthesis route for preparing the selenium-containing isoxazolamines represented by general formula I-a, I-b, I-c, I-d, I-e, and I-f, the different substituted 2,2′-diselenylbisbenzaldehydes can respectively react with different 3-amino-2,6-piperidinedione or 3-amino-1,4-dihydropyridine-2-(1H)-one or 3-amino-1-adamantanol or 2-benzothiazolamine or 3-amino-2,5-pyrroledione to afford the corresponding imine intermediates, which can produce the desired selenium-containing isoxazolamines via the reductive amination;
in the third synthesis route for preparing the tetravalent selenium-containing isoxazolamines represented by general formula I-a, I-b, I-c, I-d, I-e, and I-f, the benzisoselenidazole derivatives can be direct oxidated to the desired products with [O⁻] reagents.

6. A method of treating a disease of autoimmune diseases, neurological degenerative diseases, hematological tumors, solid tumors, myelofibrosis, and acute/chronic graft-versus-host response which are caused by the overexpression of TNF-α, comprising administering to a subject a therapeutically or prophylactically effective amount of a selenium-containing isoxazolamine derivative of claim 1, a pharmaceutically acceptable salt, a solvate, a polymorph, a stereoisomer, an isotopic compound, or a metabolite thereof.

7. A method of treating a disease of neurological degenerative diseases, hematological tumors, solid tumors, tissue ischemia-reperfusion injury, acute renal failure, and aging diseases which are caused by abnormal ferroptosis-like cell death, comprising administering to a subject a therapeutically or prophylactically effective amount of a selenium-containing isoxazolamine derivative of claim 1, a pharmaceutically acceptable salt, a solvate, a polymorph, a stereoisomer, an isotopic compound, or a metabolite thereof.

8. A method of treating a disease, symptom or disorder caused by the overexpression of TNF-α and/or abnormal ferroptosis-like cell death, wherein the method comprises administering to a subject a therapeutically or prophylactically effective amount of a substance selected from the group consisting of general formula (I) and the pharmaceutically acceptable salt thereof according to claim 1.

9. (canceled)

10. The method according to claim 7, wherein the autoimmune diseases include myelofibrosis, acute/chronic graft-versus-host response disease, rheumatoid arthritis, inflammatory bowel disease, diabetes, psoriasis, mandatory spondylitis, leprosy nodular erythema, and other infectious diseases such as HBV, HCV, HIV; the neurodegenerative diseases include Alzheimer's disease, dementia, multiple sclerosis, motor neuron disease; the blood tumor refers to multiple bone marrow tumor, myelodysplastic syndrome; the solid tumor refers to liver cancer, kidney cancer, gastric cancer, colon cancer, ovarian cancer, pancreatic cancer, prostate cancer, breast cancer, melanoma, and cerebral glioblastoma; the tissue ischemic reperfusion injury refers to stroke, coronary heart disease, myocardial infarction, pulmonary embolism, and acute coronary syndrome.
